(19) United States
(12) Patent Application Publication
(10) Pub. No.: US 2020/0338012 A1
(43) Pub. Date: Oct. 29, 2020

(54) APIXABAN TRANSDERMAL DELIVERY SYSTEM AND USES THEREOF

(71) Applicant: TAHO Pharmaceuticals Ltd., Taipei City (TW)

(72) Inventors: Catherine LEE, Happy Valley, OR (US); Hsueh-Hsien HSU, New Taipei City (TW); Tachien LU, New Taipei City (TW)

(21) Appl. No.: 16/857,304

(22) Filed: Apr. 24, 2020

Related U.S. Application Data
(60) Provisional application No. 62/839,173, filed on Apr. 26, 2019.

(51) Int. Cl. A61K 9/70; A61K 31/437

(52) U.S. Cl. CPC A61K 9/7061; A61K 31/437

(57) ABSTRACT

The present disclosure provides an apixaban transdermal patch for treatment of thrombosis and related disorders. The transdermal patch may be administered on a daily basis or a period of 2 days, 3 days, 4 days, 5 days, 6 days or 7 days. The apixaban transdermal patch preferably comprises a drug-containing layer, or a reservoir layer that comprises apixaban or a pharmaceutically acceptable salt thereof at 0.1% to about 80% by weight of the drug-containing layer.

### CROSS-REFERENCE TO PRIOR APPLICATION

This application claims priority to and the benefit thereof from U.S. Provisional Application No. 62/839,173, filed Apr. 26, 2019, titled “APIXABAN TRANSDERMAL DELIVERY SYSTEM AND USES THEREOF,” the entirety of which is hereby incorporated herein by reference.

### 1. FIELD

The present invention relates to an apixaban transdermal patch. More specifically, the present invention relates to an apixaban transdermal patch for transdermal/topical administration, method of making and uses thereof.

### 2. BACKGROUND

A transdermal patch is a small adhesive bandage that contains the drugs to be delivered. A simple type of such transdermal patches is an adhesive monolith including a drug-containing layer or a reservoir layer disposed on a backing. The reservoir layer is typically formed from a pharmaceutically acceptable pressure sensitive adhesive, which can provide adhesion to the body surface. In some cases, the reservoir layer can be formed from a non-adhesive material, the body-contacting surface of which is provided with a thin layer of a suitable adhesive, which can also contain the drug being delivered. The rate at which the drug is administered to the patient from these patches varies. Some patches can be multilaminate and can include a drug release-rate-control membrane disposed between a drug containing layer or a drug reservoir layer and the body-contacting adhesive. This membrane, by decreasing the in vitro release rate of drug from the patch, is used to reduce the effect of variations in skin permeability.

Although the transdermal delivery of therapeutic agents has been the subject of intense research and development for over 30 years, only a relatively small number of drugs are suitable for transdermal delivery due to the fact that human skin is an excellent barrier. Various techniques have been explored to enhance the permeation of drugs that are not otherwise suitable for transdermal delivery.

Although antithrombotic medications are typically in the form of oral tablets and injectables, transdermal patches offer an alternative form of administration. Specifically, compared to tablets, transdermal patches provide reduced dosing frequency, prolonged therapeutic duration, avoidance of gastrointestinal absorption as well as hepatic first-pass metabolism, minimized fluctuation in plasma drug concentrations, noninvasive administration with advantages over the oral route of administration, easy termination of drug administration by simply removing the patch from the skin and improved patient compliance. Prausnitz et al., Transdermal drug delivery, Nat. Biotechnology, 2008 November; 26(11): 1261-1268. Gaikwad A., Transdermal drug delivery system: Formulation aspects and evaluation, Comprehensive Journal of Pharmaceutical Sciences. February 2013, Vol. 1(1), pp. 1-10. Paude et al., Challenges and opportunities in dermal/transdermal delivery, NIH Public Access; 2010 Jul. 1(1): 109-131. Wiechers, et al., Formulating for Efficacy, International Journal of Cosmetic Science, 2004, 26, 173-183.

With respect to improved patient compliance, the transdermal patch is particularly beneficial to patients in comparison to tablets or injectables. A patient can easily forget whether he or she has already taken a capsule or tablet whereas, in contrast, a patient can easily tell whether a new transdermal patch has been applied, making it easier for a patient to follow required dosing regimen. Dosing regimen compliance in the patients with thrombosis is particularly important since thrombosis requires lifelong treatment. The simplified drug regimen would substantially improve the quality of life of the patients as well as their caregivers. Because the side effects associated with administration of antithrombotic medications can significantly impact a patient's health and well-being, alternatives to the current therapies are needed. Apixaban is one of the most upstream anti-thrombotic drug that blocks the conversion of prothrombin to thrombin. Fibrinogen in an environment with reduced thrombin will be converted less to fibrin for clot formation. Apixaban when administered orally, though safer than warfarin, still causes gastrointestinal (“GI”) track, including upper GI, lower GI and rectal bleeding. Oral administration of apixaban is, however, sometimes undesirable.

Sometimes a patient may have difficulty swallowing pills, or remembering to take the oral doses at all. Patient compliance has been a concern for treatments such as thrombosis. Since thrombosis does not cause symptoms until it is too late. Thus, it is desirable to have transdermal apixaban delivery patches that can continually deliver apixaban over an extended period of time. For delivery to humans, better designs to improve apixaban permeation will be required. Thus, a transdermal apixaban delivery device with adequate drug loading and sufficient flux is needed for effective therapy of ailments such as hypertension or prophylaxis of migraine. There is a need for improved delivery of apixaban, especially sustained transdermal delivery over a period of time.

Apixaban is a poor candidate for traditional transdermal delivery. Providing an apixaban transdermal system faces many technology barrier because of its physicochemical properties. With a water solubility of 0.0679 mg/ml, melting point of 237-238° C., polar surface area of 110.76 Å², it has been a huge hurdle to provide an effective transdermal patch comprising Apixaban. It is therefore, important to provide a transdermal system that are tailored for apixaban delivery taking into consideration of its physicochemical properties.

### 3. SUMMARY

The present disclosure provides a transdermal patch comprising: a drug-containing layer, or a reservoir layer, and a backing layer, wherein the drug-containing layer or the reservoir layer comprises apixaban or a pharmaceutically acceptable salt thereof.

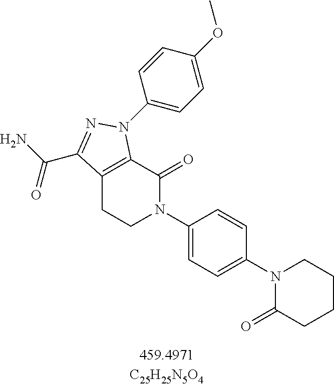 [SMILES: COc1ccc(-n2nc(C(N)=O)c3c2C(=O)N(c2ccc(N4CCCCC4=O)cc2)CC3)cc1]

In one embodiment, the patch is stable in room temperature for more than 1 month with less than 5% impurities.

In certain embodiments, the drug-containing layer, or the reservoir layer of the present dermal patch (or the present composition) comprises apixaban (free base). In certain embodiments, the drug-containing layer, or the reservoir layer of the present dermal patch (or the present composition) comprises an ester of apixaban. In one embodiment, the compounds of the present disclosure is a salt. In certain embodiments, the drug-containing layer, or the reservoir layer of the present dermal patch (or the present composition) comprises apixaban.

Provided herein is a transdermal apixaban delivery devices and formulations that deliver apixaban base or a salt thereof in a therapeutically effective amount. Since transdermal delivery of apixaban has higher bioavailability than oral delivery, transdermal doses of 1 mg, 2.5 mg, 5 mg, 10 mg, 15 mg, 20 mg, 40 mg and 80 mg apixaban free base equivalent per day (adjusted for free base form and oral bioavailability) should produce the therapeutic effect like those of the orally delivered apixaban. The formulations have low irritation potential and contain sufficient drug to support one-day or multi-day delivery while maintaining reasonable adhesiveness.

In one aspect, a transdermal apixaban delivery system is provided to provide health benefit to a subject in need thereof. The system includes a backing layer, a reservoir layer containing apixaban drug disposed proximally relative to the backing layer for one day or multiple day use. The reservoir layer has a polymeric composition containing an amount of apixaban or a pharmaceutically acceptable salt thereof sufficient for at least one-day delivery, or multiple-day delivery. In one embodiment, the delivery system comprises a rate-control adhesive layer disposed proximally to the body surface (e.g., skin) relative to the reservoir layer. The rate-control adhesive layer disposed proximally relative to the reservoir layer to control the rate of apixaban delivery to the subject. In one embodiment, the apixaban drug is apixaban free base.

In another aspect, provided herein is a method of making a transdermal apixaban delivery system. In one aspect, provided herein is a method of using the transdermal apixaban delivery system. The method for making a transdermal patch for administering apixaban to a subject includes disposing a reservoir layer to the subject proximally relative to a backing layer. The reservoir layer comprises a polymeric composition that comprises an amount of apixaban or a pharmaceutically acceptable salt thereof sufficient for at least one-day delivery, or multiple-day delivery. In one embodiment, the delivery system further comprises a rate-control adhesive layer disposed proximally to the body surface relative to the reservoir layer. The rate-control adhesive layer is disposed proximally relative to the reservoir layer to control the rate of apixaban delivery to the subject. In one embodiment, the apixaban drug is apixaban free base.

A transdermal delivery device is provided with an effective amount of apixaban. In one embodiment, the apixaban is completely dissolved into a drug reservoir layer matrix layer. Once applied on a subject's body surface, the device can stay adhesively to the body surface over an extended period of time during which apixaban is to be delivered by the device. In certain embodiments, the device delivers apixaban for a period of 1 day, 3 days, 7 days or 14 days.

In one embodiment, the transdermal delivery of apixaban results in lower adverse events than with oral delivery. Further, a transdermal patch allows a more steady sustained delivery than doses taken orally at time intervals hours apart. The patch leads to improved compliance in the patients.

In one embodiment, the transdermal system delivery of a therapeutic dose of apixaban (about 1.25 mg to 20 mg per day) from a thin, flexible patch. In certain embodiments, the patch is about 5 to 125 cm² in size. In one embodiment, the patch is about 40 cm² in size. In one embodiment, the length of the patch is about 5-6 cm. In one embodiment, the length of the patch is about 6.3 cm. In one embodiment, the patch is about 4 mm to 200 μm thick. In certain embodiment, the patch delivers the drug for a duration of 12 hours to 96 hours. In certain embodiments, the average input range of the patch is 0.1-11 μg/cm²hr. In certain embodiments, the average input is about 5.1 μg/cm²hr. In certain embodiments, the drug loading is 1%, and 3%.

In certain embodiments, the transdermal system delivers 5 μg-20 mg of apixaban per day. In one embodiment, the transdermal system comprises a composition comprising apixaban. In certain embodiments, the transdermal system delivers a composition comprising apixaban that has a half-life of 1-10 hours. In certain embodiments, the patch delivers a composition comprising apixaban that has a melting point of less than 200° C. In certain embodiments, the partition coefficient of the composition comprising apixaban is 1-4. In certain embodiments, the composition comprising apixaban has an aqueous solubility of greater than lmg/ml. In certain embodiments, the composition comprising apixaban has a pH of 5-9. In certain embodiments, the skin permeability coefficient of the composition comprising apixaban is greater than 0.5×10-3 cm/hr. The patch of the present disclosure is non-irritating and non-sensitizing. The composition comprising apixaban has a low oral bioavailability.

The therapeutic dose requirement for transdermal administration has been determined by adjusting the prescribed oral dose of apixaban fumarate with the oral bioavailability and the molecular weight difference of the salt to that of the free base (which oral bioavailability and molecular weight difference are known to those skilled in the art).

In an aspect, certain patches are provided that can deliver apixaban base systemically at a therapeutically effective rate for providing therapeutic benefits for ailments without using a significant amount of, and even without any, permeation enhancer. In certain embodiments, rate-control is provided to slow down the flux by including a rate-control in-line adhesive and/or rate-control tie layer(s).

In one embodiment, a patch is applied on the body surface of a patient for use to render therapeutic benefits for ailments such as thrombosis. As used herein, “treatment” or “therapeutic benefit” includes relief or reduction of symptoms and prophylaxis of symptoms. In one embodiment, the apixaban transdermal delivery systems is used for postoperative administration such as for prophylaxis to reduce the risk of thrombosis.

In another aspect, a method is provided to load a therapeutically effective amount of apixaban into the drug reservoir layer of the transdermal patch that can be worn for an extensive period of time, such as 3 days, 4 days, or 7 days. Patches that can be used for such extensive periods of time would increase patient compliance and would reduce a caregiver's burden.

In one aspect, the present transdermal device with apixaban will address some of the challenges to providing optimal apixaban therapy. A 3 -day, 4-days, or 7-day transdermal delivery system, in addition to reducing caregiver burden and improving dosing compliance, should result in less gastrointestinal exposure compared to oral administration and could decrease the incidence of gastrointestinal side effects associated with peripheral cholinergic stimulation. Transdermal flux rates which produce gradually increasing plasma levels over several days may reduce the need for dosing titration and simplify the dosing regimen. An ability to achieve and tolerate higher apixaban levels or more rapid dose titration would be expected to result in greater efficacy, earlier onset of symptomatic improvement (for symptomatic ailments), or both.

In one embodiment, the transdermal delivery system comprises a matrix layer. In one embodiment, the transdermal delivery system comprises a reservoir layer.

In one embodiment, the reservoir layer comprises a hydrogel.

In one embodiment, the matrix layer comprises an EVA polymer.

In one embodiment, the matrix layer comprises a PIB polymer.

In one embodiment, the matrix layer comprises an acrylic-based polymer and a silicone adhesive.

In one embodiment, the transdermal delivery system is prepared using a hot melt method.

In one embodiment, the transdermal delivery system comprises a permeation enhancer.

In one embodiment, the transdermal delivery system comprises an adhesive and lactic acid. In one embodiment, the transdermal delivery system comprises an adhesive and acrylic acid. In one embodiment, the transdermal delivery system comprises an adhesive and erucic acid/DCM.

In one embodiment, the hydroxyl functional group contains acrylic-based polymer is an acrylates copolymer.

In one embodiment, the one or more acrylic-based polymers provides a solubility of no greater than about 10% for apixaban or a pharmaceutically acceptable salt thereof.

In one embodiment, the apixaban or a pharmaceutically acceptable salt thereof is in an amount ranging from about 2% to about 15% by weight (wt %) relative to total weight of the drug-containing layer, or the reservoir layer.

In one embodiment, the apixaban or a pharmaceutically acceptable salt thereof is in an amount ranging from about 5% to about 10% by weight (wt %) relative to total weight of the drug-containing layer, or the reservoir layer.

In one embodiment, the apixaban or a pharmaceutically acceptable salt thereof is about 8% by weight (wt %) relative to total weight of the drug-containing layer, or the reservoir layer.

In one embodiment, the hydroxyl functional group containing acrylic-based polymer is sourced from a polymer solution of acrylates copolymer comprising 2-hydroxyethyl acrylate or from a polymer solution of acrylates copolymer comprising vinyl acetate and 2-hydroxyethyl acrylate.

In one embodiment, the drug-containing layer, or the reservoir layer further comprises a permeation enhancer.

In one embodiment, the permeation enhancer is an alcohol, a fatty acid, a fatty alcohol, a pharmaceutically acceptable solvent, a pharmaceutically acceptable surfactant, or combinations thereof.

In one embodiment, the permeation enhancer is aliphatic alcohols, fatty acids having chain of 8 to 20 carbons, fatty acid esters, alcohol amines, polyhydric alcohol alkyl ethers, polyoxyethylene alkyl ethers, glycerides, middle-chain fatty acid esters of polyhydric alcohols having chain of 8-20 carbon atoms, alkyl esters having chain of 1-6 carbon atoms, acylated amino acids, pyrrolidone, pyrrolidone derivatives, ethoxylated fatty alcohols, pharmaceutically acceptable surfactants or a combination thereof.

In one embodiment, the permeation enhancer is 1,2-propyleneglycol, a polysorbate, hydroxypropyl cellulose (HPC), or combinations thereof.

In one embodiment, the permeation enhancer comprises polysorbate 80.

In one embodiment, the permeation enhancer is in an amount ranging from about 5% to about 30% by weight (wt %) relative to total weight of the drug-containing layer, or the reservoir layer.

In one embodiment, the permeation enhancer is in an amount ranging from about 5% to about 15% by weight (wt %) relative to total weight of the drug-containing layer, or the reservoir layer.

In one embodiment, the permeation enhancer is about 10% by weight (wt %) relative to total weight of the drug-containing layer, or the reservoir layer.

In one embodiment, the permeation enhancer provides a solubility of greater than about 20 mg/mL for apixaban or a pharmaceutically acceptable salt thereof.

In one embodiment, the transdermal patch further comprises an organic solvent.

In one embodiment, the organic solvent is 1,3-Dimethyl-2-imidazolidinone (DMI), dichloromethane (DCM), or a combination thereof.

In one embodiment, the organic solvent is in an amount ranging from about 5% to about 30% by weight (wt %) relative to total weight of the drug-containing layer, or the reservoir layer.

In one embodiment, the organic solvent is in an amount ranging from about 10% to about 20% by weight (wt %) relative to total weight of the drug-containing layer, or the reservoir layer.

In one embodiment, the organic solvent is about 15% by weight (wt %) relative to total weight of the drug-containing layer, or the reservoir layer.

In one embodiment, the drug-containing layer further comprises a crystallization inhibitor.

In one embodiment, the drug-containing layer further comprises an antioxidant.

In one embodiment, the transdermal patch further comprises a protective layer.

In one embodiment, the transdermal patch provides a flux rate of more than about 0.5 μg/cm².hr and less than about 20 μg/cm².hr for up to about 30 hours.

In one embodiment, lag time for the transdermal patch is less than about 8 hours.

Provided in the present disclosure is a method for treating a neurological disorder comprising the step of applying the disclosed transdermal patch to a human subject in need thereof.

In one embodiment, the transdermal patch is applied to the human subject for a period of about 24 hours.

In certain embodiments, about 1 mg to about 3 mg, about 3 mg to about 5 mg, about 5 to about 10 mg, about 10 mg to 12 mg, about 12 mg to about 15 mg, about 15 mg to about 20 mg of apixaban is delivered from the transdermal patch to the human subject daily.

In one embodiment, about 3 mg to about 12 mg of apixaban is delivered from the transdermal patch to the human subject daily.

Provided in this disclosure is a pharmaceutical composition for topical application, the composition comprising (a) apixaban or a pharmaceutically acceptable salt thereof, and (b) two or more acrylic-based polymers.

In one embodiment, the apixaban or a pharmaceutically acceptable salt thereof is in an amount ranging from about 2% to about 15% by weight (wt %) relative to total weight of the pharmaceutical composition.

In one embodiment, the apixaban or a pharmaceutically acceptable salt thereof is in an amount ranging from about 5% to about 10% by weight (wt %) relative to total weight of the pharmaceutical composition.

### 4.1 BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-B. (A) apixaban PK profile: plasma time course of apixaban on days 1 and 7 following twice daily administration of apixaban 2.5, 5, 10 or 20 mg. (B) Repeated dose on healthy volunteer on apixaban oral, daily 2.5 mg, 5 mg and 10 mg BD.

FIG. 2 shows the results for in vitro solution permeation data on various formulations.

FIGS. 3A-B show apixaban oral bioavailability. (A) Summary of pharmacokinetic measures/parameters for apixaban following intravenous administration. (B) Intravenous administration of apixaban. This shows that the oral tablet has a bioavailability of 50%. FIG. 4 shows in vitro permeation data on various formulations.

FIGS. 5A-B show sides effect vs. apixaban exposure. It is possible to reduce major bleeding with lower apixaban exposure. ISTH major bleeding events increased with an increase in apixaban exposure (AUC_{SS}) at the dose level studied. Probability of major bleeding by AUC_{SS} shown in (A) linear logistic regression model and (B) Cox PH regression model indicating a predicted probability of an event within one year after controlling for other covariates.

FIG. 6 shows risk management with patch approach. Major bleeding side effect is Cmax related.

FIG. 7 shows apixaban solubility at room temperature.

FIG. 8 shows in vitro permeation data.

FIG. 9 shows in vitro permeation data on hydrogel formulations

FIG. 10 shows in vitro permeation data on COOH functional group acrylic matrix patch formulations.

FIG. 11 shows in vitro permeation data on OH functional group acrylic matrix patch formulations.

FIG. 12 shows in vitro permeation data on formulations for 7-day matrix patch.

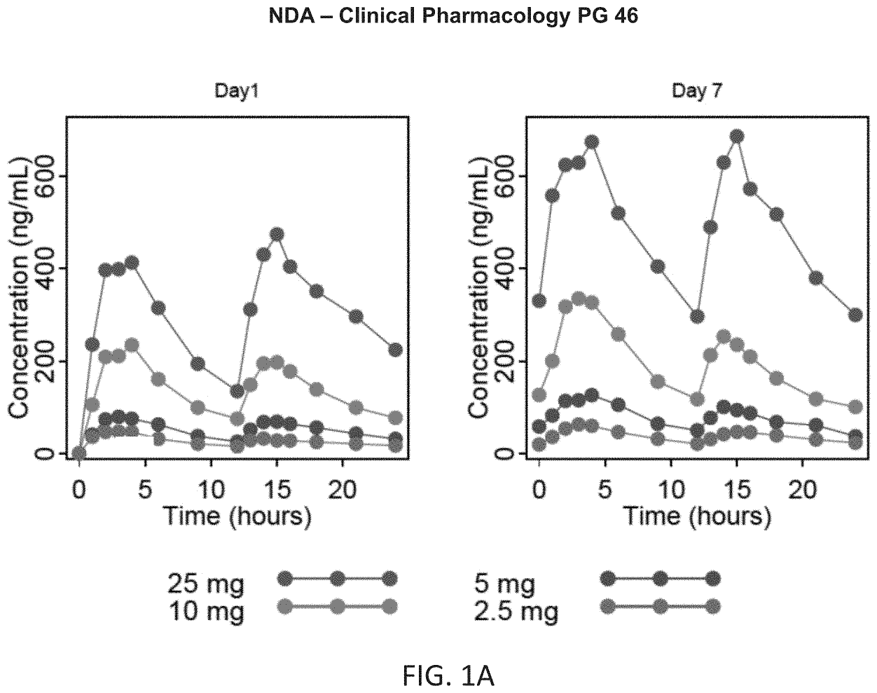

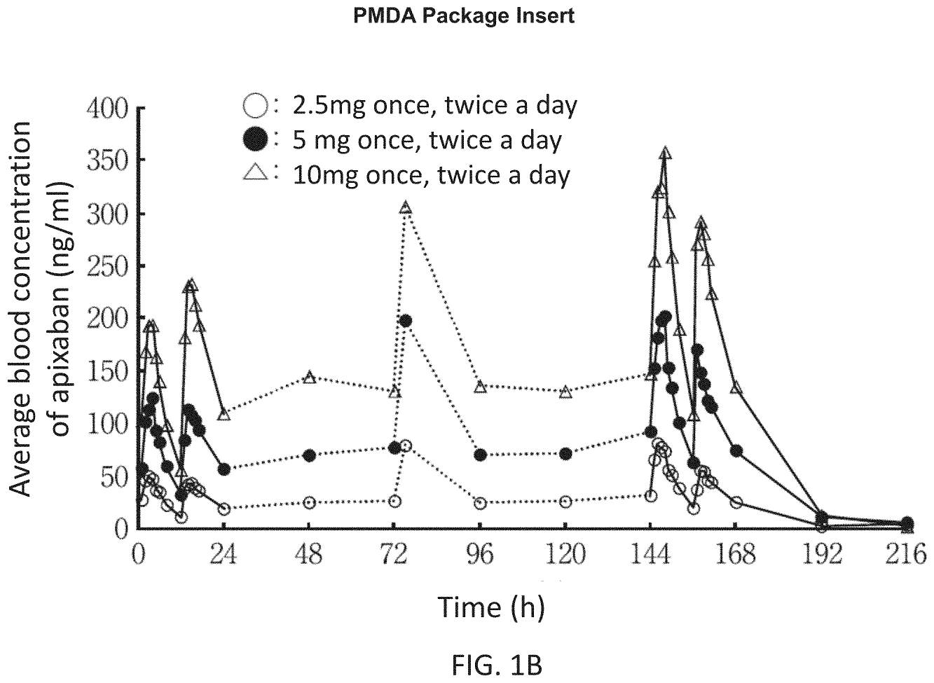

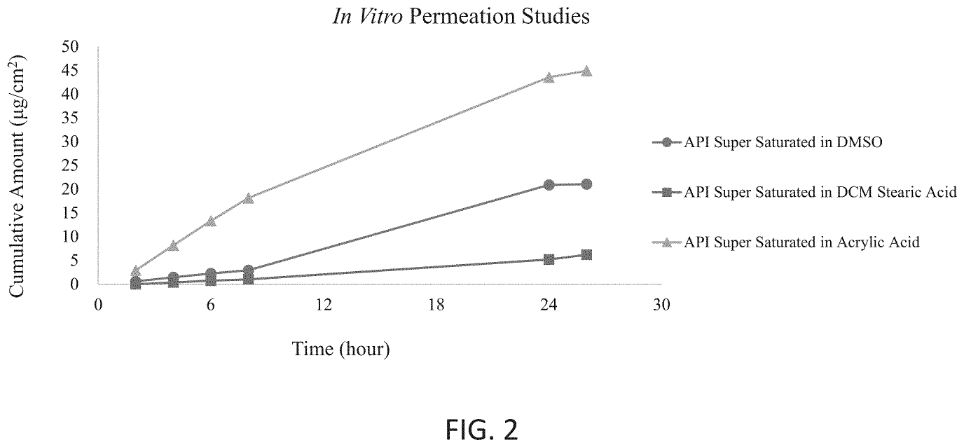

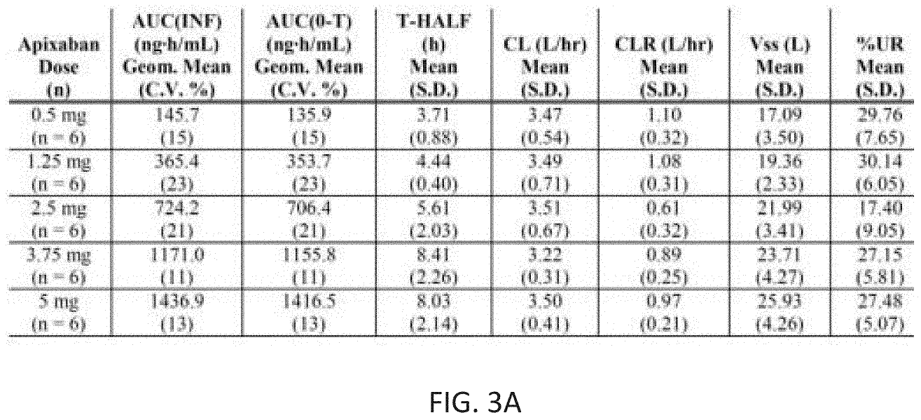

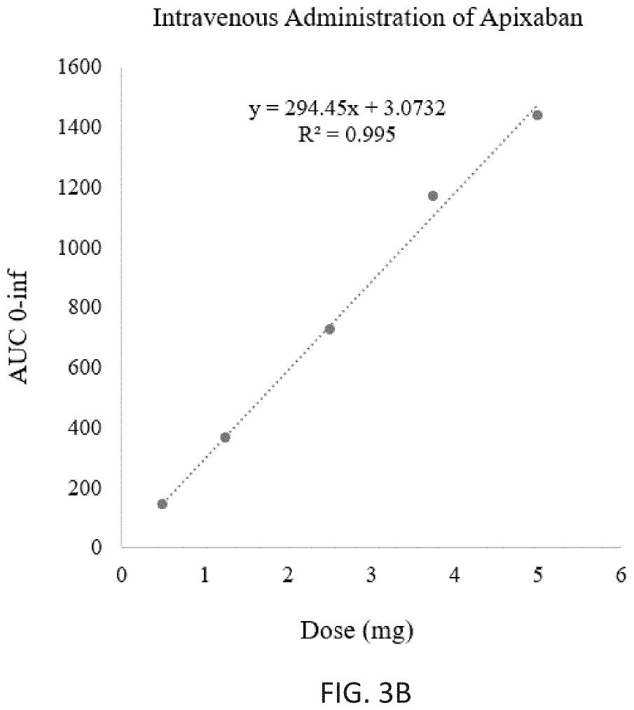

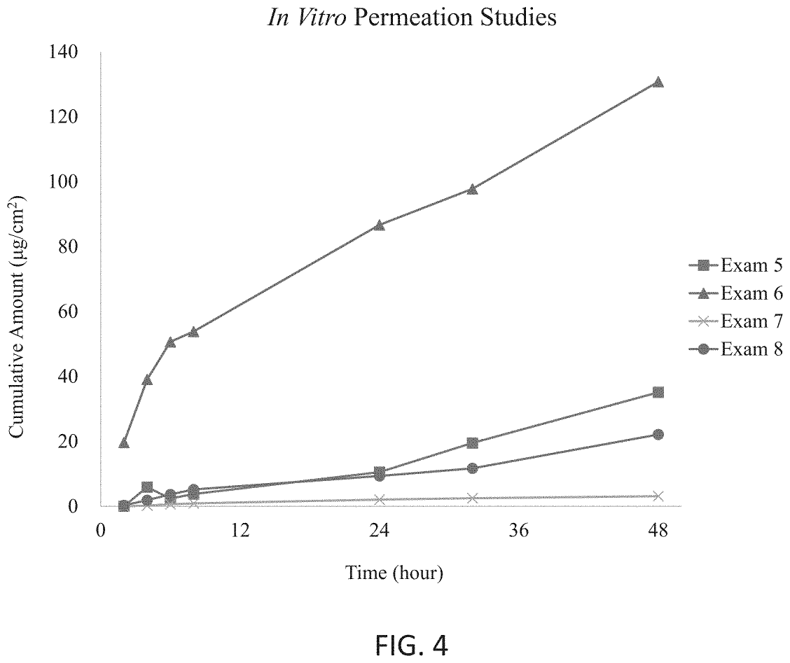

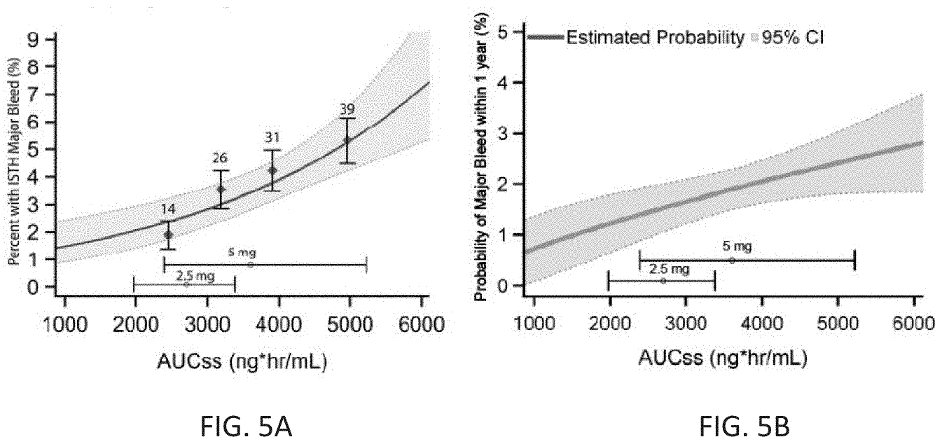

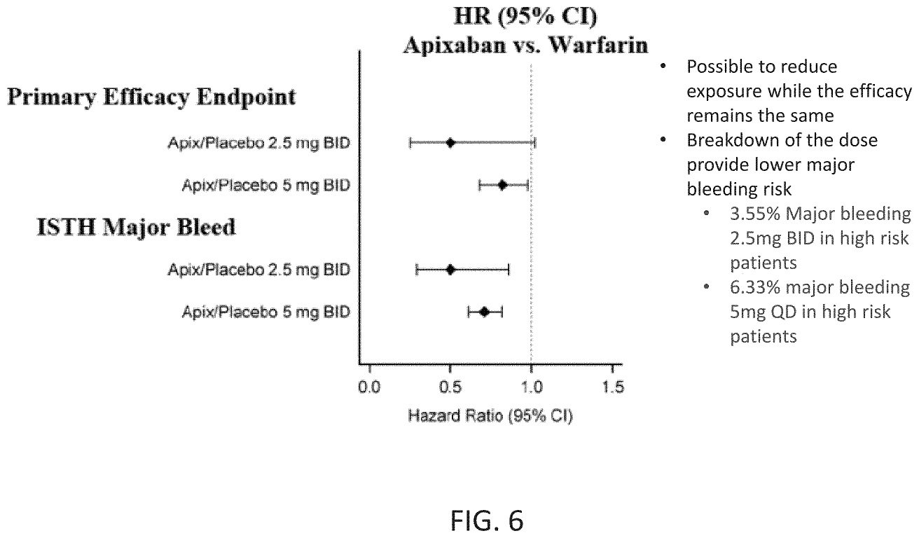

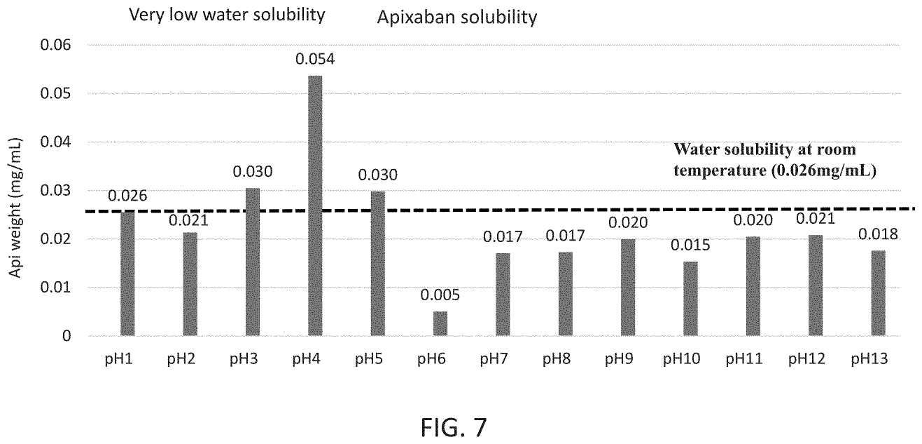

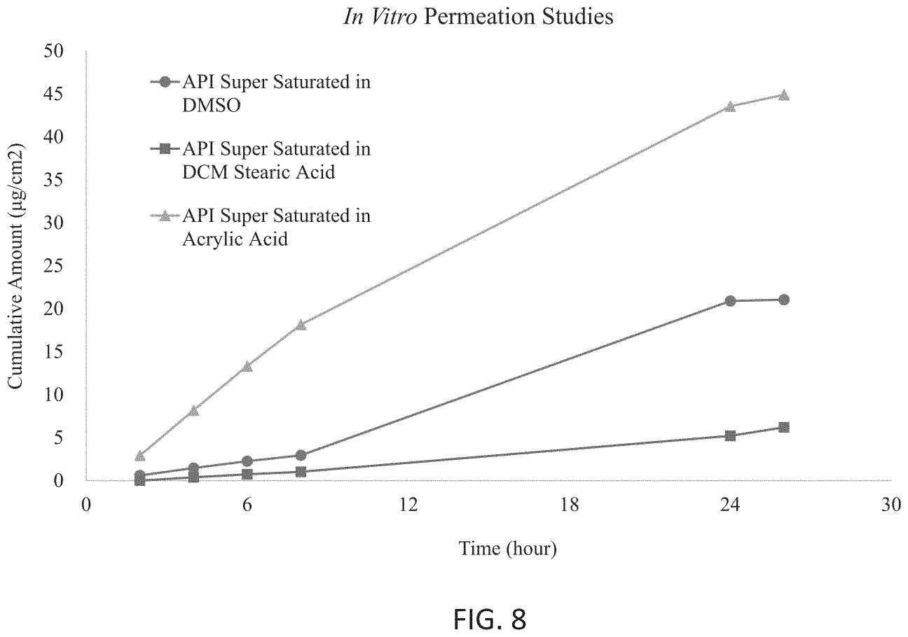

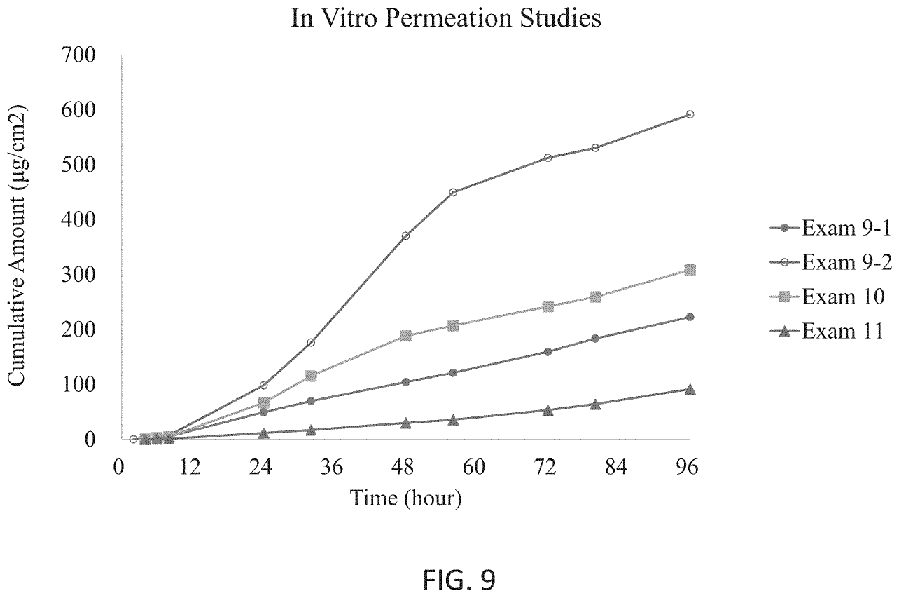

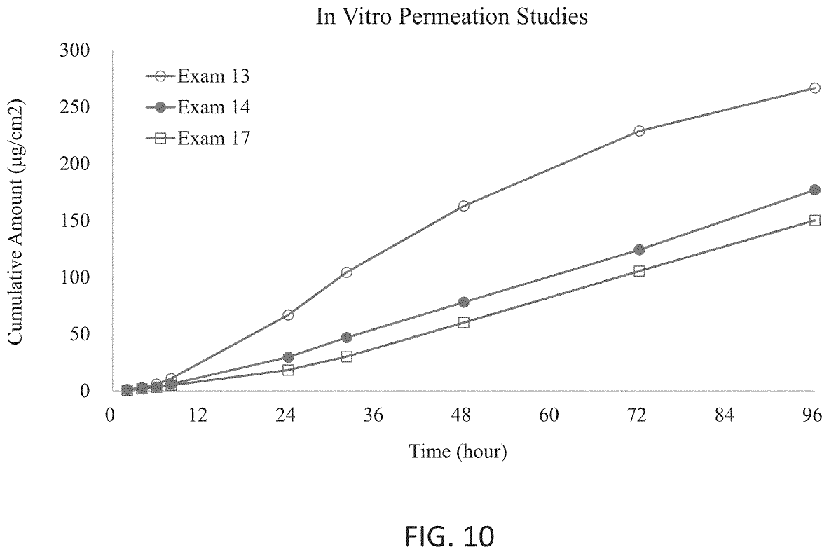

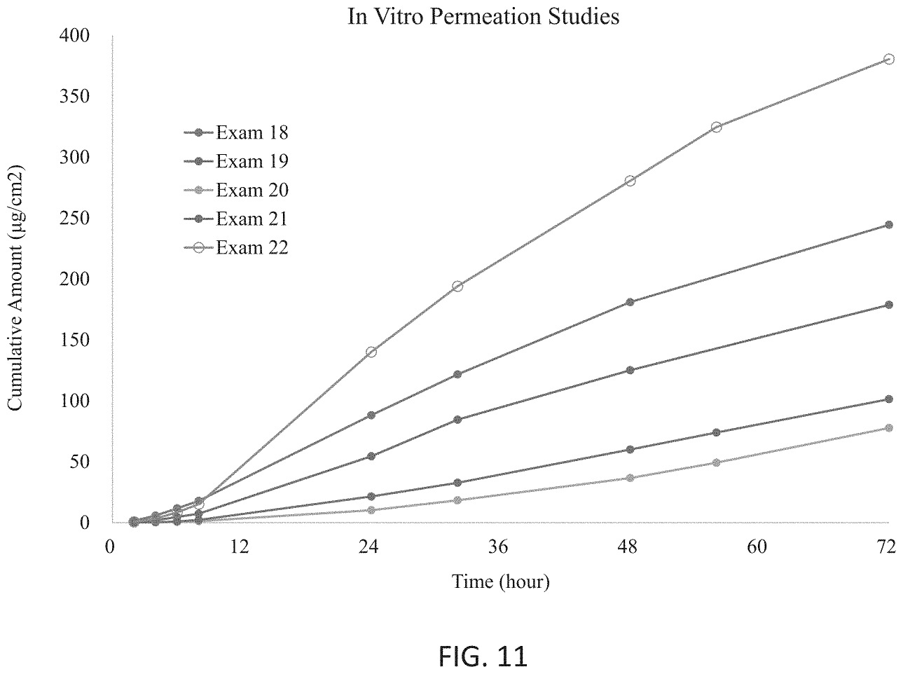

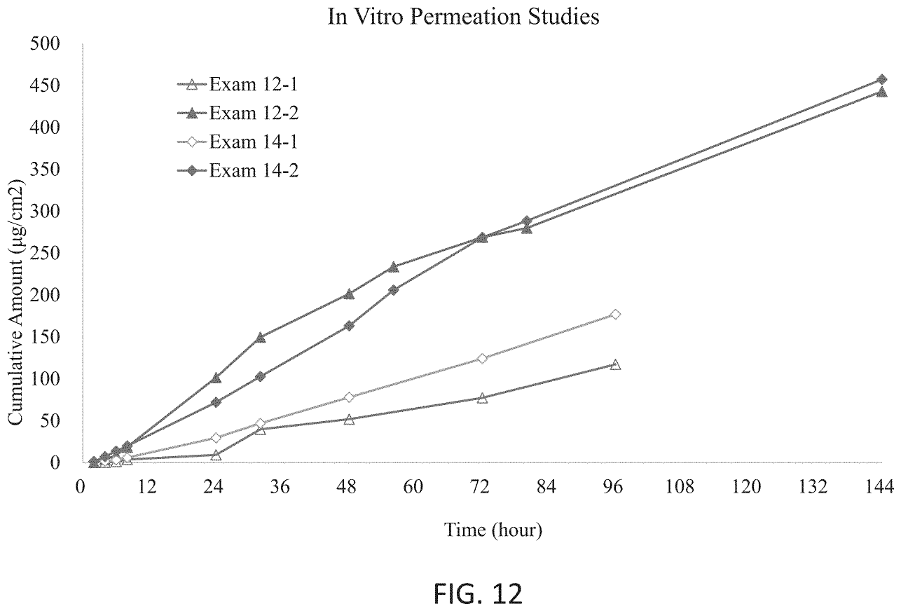

### 4.2 DEFINITIONS

As used herein, the singular forms “a”, “an” and “the” include plural referents unless the context clearly indicates otherwise. Thus, for example, reference to “an ingredient” includes mixtures of ingredients, reference to “an active pharmaceutical agent” includes more than one active pharmaceutical agent, and the like.

The terms “active agent”, “pharmacologically active agent” and “drug” are used interchangeably herein to refer to a chemical material or compound that includes a desired pharmacological, physiological effect and include agents that are therapeutically effective. The terms also encompass pharmaceutically acceptable, pharmacologically active derivatives and analogs of those active agents specifically mentioned herein, including, but not limited to, salts, esters, amides, prodrugs, active metabolites, inclusion complexes, enantiomers S(−) or R(+), analogs of the active agent (e.g., apixaban).

The compounds of the present disclosure may be a salt. As used herein, a “salt” is a salt of the present compound which has been modified by making acid or base, salts of the compounds. The salt may be pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately treating a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The present methods also encompass administering a physiologically functional derivative of the present compound. As used herein, the term “physiologically functional derivative” refers to a compound (e.g., a drug precursor) that is transformed in vivo to yield the present compound or its active metabolite, or a pharmaceutically acceptable salt, hydrate or solvate of the compound. The transformation may occur by various mechanisms (e.g., by metabolic or chemical processes), such as, for example, through hydrolysis. Prodrugs are such derivatives, and a discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

As used herein, the term “about” as a modifier to a quantity is intended to mean +or −5% inclusive of the quantity being modified. As used herein, “wt %”, “% w/w” or “% (w/w)” refer to % by weight of the composition.

As used herein, the term “matrix” refers to a solid, or semi-solid substance, such as, for example, a polymeric material, adhesive or gel, that has capacity to hold a beneficial agent or drug for transdermal drug delivery. In some cases the matrix can also hold liquid. The matrix serves as a repository (or carrier) in which the beneficial agent or drug is carried (contained) and may be porous. For the sake of convenience, when mentioned along with ingredients, sometimes “matrix” as referred herein can include drugs or ingredients held therein.

As used herein, the term “in-line” when referring to a layer means the layer is in the direct shortest path of the drug flowing from the drug reservoir layer to the body surface. Thus an in-line adhesive of a device is an adhesive layer that is disposed in the direct shortest path between the drug reservoir layer and the body surface on which the device is placed such that the drug has to pass through the in-line adhesive to reach the body surface.

The present agent/composition may be administered therapeutically to achieve a therapeutic benefit (“treating”) or prophylactically to achieve a prophylactic benefit (“preventing”). By therapeutic benefit is meant eradication or amelioration of the disorder or condition being treated, and/or eradication or amelioration of one or more of the symptoms associated with the disorder or condition. By prophylactic benefit is meant prevention or delay of the onset of the condition, and/or prevention or delay of the onset of one or more of the symptoms associated with the condition. In certain embodiments, an effective amount of the present agent/composition to be administered prevents the condition from developing or being exacerbated into more serious conditions.

“Treating” or “treatment” of a state, disorder or condition includes: (1) preventing or delaying the appearance of clinical symptoms of the state, disorder, or condition developing in a person who may be afflicted with or predisposed to the state, disorder or condition but does not yet experience or display clinical symptoms of the state, disorder or condition; or (2) inhibiting the state, disorder or condition, i.e., arresting, reducing or delaying the development of the disease or a relapse thereof (in case of maintenance treatment) or at least one clinical symptom, sign, or test, thereof; or (3) relieving the disease, i.e., causing regression of the state, disorder or condition or at least one of its clinical or sub-clinical symptoms or signs. The benefit to a subject to be treated is either statistically significant or at least perceptible to the patient or to the physician.

An effective amount of an agent/drug refers to a therapeutically effective amount or a prophylactically effective amount. A “prophylactically effective amount” refers to an amount effective, at dosages and for periods of time necessary, to achieve the desired prophylactic result. In certain embodiments, since a prophylactic dose is used in subjects prior to or at an earlier stage of a disorder, the prophylactically effective amount is less than the therapeutically effective amount. In certain embodiments, the prophylactically effective amount is similar to, identical to, or more than, the therapeutically effective amount. A therapeutically effective amount of a drug is an amount effective to demonstrate a desired activity of the drug. A therapeutically effective amount may vary depending on the compound, the disorder and its severity and the age, weight, physical condition and responsiveness of the subject to be treated. In certain embodiments, the drug-containing layer, or the reservoir layer or the present composition further comprises a pharmaceutically acceptable carrier, vehicle, excipient and/or diluent.

The term “transdermal patch”, or “dermal patch”, is intended to refer to a self-contained, discrete dosage form that, when applied to skin, is designed to deliver the drug(s) through the skin into systemic circulation. Some important characteristics of a transdermal patch include flux rate, lag time and stability. Flux rate relates to the rate at which the transdermal patch delivers apixaban. Lag time relates to the time required for apixaban blood concentration to reach steady state after application of the transdermal patch. Lag time preferably matches apixaban metabolic rate in order to minimize fluctuations in blood concentration between applications of successive transdermal patches. Lastly, stability relates to the amount of impurities that develops within the transdermal patch while in storage.

### 5. DETAILED DESCRIPTION

The present disclosure provides a transdermal patch containing apixaban, or a pharmaceutically acceptable salt, derivative, or solvate thereof, as an active agent. In certain embodiments, the transdermal patch is for daily administration with minimal apixaban blood concentration fluctuations. In certain embodiments, the transdermal patch provides high flux of apixaban and low crystallization of the active agent.

The present disclosure also provides a topical composition containing apixaban, or a pharmaceutically acceptable salt, derivative, or solvate thereof, as an active agent.

The present disclosure provides methods and compositions (e.g., a transdermal patch, a topical composition, etc.) for treating or preventing thrombosis. In certain embodiments, the disclosure provides methods and compositions for treating or preventing left ventricular thrombus, atrial fibrillation, acute coronary syndrome, reduce the risk of stroke and systemic embolism. In one embodiment, the methods and compositions provide treatment of subjects with nonvalvular atrial fibrillation. In certain embodiments, the methods and compositions provide prophylaxis of deep vein thrombosis, which may lead to pulmonary embolism. In certain embodiments, the subject has undergone surgery. In certain embodiments, the methods and composition reduce the risk of recurrent deep vein thrombosis and pulmonary embolism following initial therapy.

Also encompassed by the present disclosure is a method of treating or preventing thrombosis and other disorders. The method may comprise applying the present composition (e.g., a transdermal patch, a topical composition, etc.) to a subject (e.g., to an area of the skin of a subject).

### 5.1 TRANSDERMAL DELIVERY SYSTEM

The present disclosure provides a transdermal patch comprising: a drug-containing layer, or the reservoir layer, and a backing layer. In certain embodiments, the transdermal patch further comprises a protective layer.

Apixaban, is a pyrazolopyridine that is 7-oxo-4,5,6,7-tetrahydro-1H-pyrazolo[3,4-c]pyridine-3-carboxamide substituted at position 1 by a 4-methoxyphenyl group and at position 6 by a 4-(2-oxopiperidin-l-yl)phenyl group with the following representative structure.

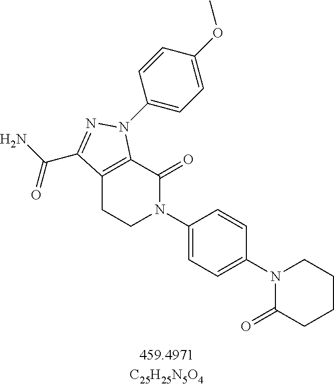 [SMILES: COc1ccc(-n2nc(C(N)=O)c3c2C(=O)N(c2ccc(N4CCCCC4=O)cc2)CC3)cc1]

In certain embodiments, apixaban is in the free base form. In certain embodiments, the drug-containing layer, or the reservoir layer of the present dermal patch (or the present composition) comprises a pharmaceutically acceptable salt of apixaban. In certain embodiments, the salt of apixaban is an acid addition salt formed by treatment with an appropriate acid, such as a hydrohalic acid, for example hydrochloric or hydrobromic acid, sulfuric acid, nitric acid, phosphoric acid, acetic acid, propanoic acid, hydroxyacetic acid, 2-hydroxypropanoic acid, 2-oxopropanoic acid, ethanedioic acid, propanedioic acid, butanedioic acid, (Z)-2-butenedioic acid, (E)-2-butenedioic acid, 2-hydroxybutanedioic acid, 2,3-dihydroxybutanedioic acid, 2-hydroxy-1,2,3-propanetricarboxylic acid, methanesulfonic acid, ethanesulfonic acid, benzenesulfonic acid, 4-methylbenzenesulfonic acid, cyclohexanesulfamic acid, 2-hydroxybenzoic acid or 4-amino-2-hydroxybenzoic acid. In one embodiment, the present dermal patch comprises the apixaban base form.

In certain embodiments, the drug-containing layer, or the reservoir layer of the present dermal patch (or the present composition) comprises an ester of apixaban.

In certain embodiments, apixaban is prepared or obtained, and further purified. In certain embodiments, apixaban may be purified by dissolving the apixaban in a chlorinated solvent, such as methylene chloride, ethylene chloride, chloroform, preferably methylene chloride, and heating the reaction mixture. The chlorinated solvent may be mixed with a second solvent, such as methanol. The solution of apixaban may then be concentrated by heating, distilling and the then cooling the reaction mixture. The pure apixaban is then isolated using a suitable solvent, such as acetone, methanol, ethyl acetate, isopropyl alcohol, methyl isobutyl ketone (MIBK), acetonitrile, isopropyl acetate, toluene, preferably methanol. The isolation step comprises filtering the product, washing the product in the suitable solvent, and then drying under vacuum.

Provided herein is a transdermal patch comprising: (i) a drug-containing layer; (ii) a matrix layer or a reservoir layer and (iii) a backing layer, wherein the drug-containing layer comprises apixaban or a pharmaceutically acceptable salt thereof and wherein the transdermal patch has an average input rate of about 2-7 μg/cm²hr.

In certain embodiments, the matrix layer or reservoir layer comprises an acrylic-based polymer, wherein the acrylic-based polymer comprises a hydroxyl functional group containing acrylic-based polymer.

In certain embodiments, the matrix layer or reservoir layer further comprises a silicone adhesive.

In certain embodiments, the matrix layer or reservoir layer comprises a polyisobutylene polymer, silicone, EVA polymer, non-acidic polyacrylate, hydrogel polymer or a combination thereof.

In certain embodiments, the acrylic-based polymer and the silicone are at a weight ratio ranging from about 5:1 to about 1:5.

In certain embodiments, the hydroxyl functional group containing acrylic-based polymer is an acrylates copolymer. In certain embodiments, the acrylic-based polymers provides a solubility of greater than 5% for apixaban or a pharmaceutically acceptable salt thereof.

In certain embodiments, the matrix layer or reservoir layer comprises one or more solvents.

In certain embodiments, the solvent is lactic acid, acrylic acid, erucic acid or a combination thereof.

In certain embodiments, the solvent is 1,2-propyleneglycol, lactic acid or a combination thereof.

In certain embodiments, the apixaban or a pharmaceutically acceptable salt thereof is in an amount ranging from about 2% to about 15% by weight (wt %) relative to total weight of the drug-containing layer.

In certain embodiments, the hydroxyl functional group containing acrylic-based polymer is sourced from a polymer solution of acrylates copolymer comprising 2-hydroxyethyl acrylate or from a polymer solution of acrylates copolymer comprising vinyl acetate and 2-hydroxyethyl acrylate.

In certain embodiments, the drug-containing layer further comprises a permeation enhancer.

In certain embodiments, the permeation enhancer is an alcohol, a fatty acid, a fatty alcohol, a pharmaceutically acceptable solvent, a pharmaceutically acceptable surfactant, or combinations thereof.

In certain embodiments, the permeation enhancer is selected from the group consisting of aliphatic alcohols, fatty acids having chain of 8 to 20 carbons, fatty acid esters, alcohol amines, polyhydric alcohol alkyl ethers, polyoxyethylene alkyl ethers, glycerides, middle-chain fatty acid esters of polyhydric alcohols having chain of 8-20 carbon atoms, alkyl esters having chain of 1-6 carbon atoms, acylated amino acids, pyrrolidone, pyrrolidone derivatives, ethoxylated fatty alcohols, pharmaceutically acceptable surfactants or a combination thereof.

In certain embodiments, the permeation enhancer is 1,2-propyleneglycol, a polysorbate, hydroxypropyl cellulose (HPC), or combinations thereof.

In certain embodiments, the permeation enhancer comprises polysorbate 80.

In certain embodiments, the permeation enhancer is in an amount ranging from about 5% to about 30% by weight (wt %) relative to total weight of the drug-containing layer.

In certain embodiments, the permeation enhancer provides a solubility of greater than about 20 mg/mL for apixaban or a pharmaceutically acceptable salt thereof.

In certain embodiments, transdermal patch further comprising an organic solvent.

In certain embodiments, the organic solvent is 1,3-Dimethyl-2-imidazolidinone (DMI), dichloromethane (DCM), or a combination thereof.

In certain embodiments, the solvent is in an amount ranging from about 5% to about 30% by weight (wt %) relative to total weight of the drug-containing layer.

In certain embodiments, the drug-containing layer further comprises a crystallization inhibitor.

In certain embodiments, the drug-containing layer further comprises an antioxidant.

In certain embodiments, the transdermal patch further comprising a protective layer.

In certain embodiments, the transdermal patch provides a flux rate of about 0.5 μg/cm².hr to about 20 μg/cm².hr for up to about 30 hours.

In certain embodiments, the lag time for the transdermal patch is less than about 8 hours.

In certain embodiments, the patch size is from 4 cm² to 40 cm² and the rate-control in-line adhesive is one of polyisobutylene (PIB), silicone, and polyacrylate that controls the delivery rate of apixaban at 4 to 12 mg per day.

In certain embodiments, the apixaban is dissolved or dispersed in a hot melt adhesive in the reservoir layer.

In certain embodiments, the reservoir layer includes about 5-15 wt % of permeation enhancer.

In certain embodiments, the apixaban is dissolved in a hot melt of EVA in the reservoir layer, the device having an in-line rate-control adhesive of PIB.

In certain embodiments, the transdermal patch comprises a rate-control in-line adhesive that is different from the main matrix polymer, and the patch further comprising an EVA tie layer disposed between the reservoir layer and the in-line adhesive.

In certain embodiments, the reservoir layer contains EVA with 10-80 wt % or more of vinyl acetate content.

In certain embodiments, the reservoir layer contains apixaban free base adequate for delivery for 1-3 days.

In certain embodiments, the reservoir layer contains apixaban free base adequate for delivery for 3-9 days.

In certain embodiments, the reservoir layer contains 20-30 wt % apixaban free base and 30-60 wt % EVA.

In certain embodiments, the reservoir layer is substantially free of an adhesive polymer with acidic functionality.

In certain embodiments, the device has an average apixaban flux of 5-25 mcg/(cm².h) for 1 day.

In certain embodiments, the reservoir layer contains apixaban free base adequate for delivery for 3 days or more with an average flux of a flux of 5-25 mcg/(cm².h).

In certain embodiments, the transdermal patch further comprising an adhesive overlay disposed distal to the backing layer and a protective liner disposed proximal to the in-line rate-control adhesive.

In certain embodiments, the rate-control adhesive is silicone adhesive.

In certain embodiments, the rate-control adhesive contains 1-4 wt % of an alkaline salt of an organic acid.

Provided herein is a method for making a transdermal patch for administering a apixaban to a user, comprising: (a) disposing a reservoir layer proximally relative to a backing layer; and (b) forming the reservoir layer, which contains a polymeric composition containing an amount of apixaban free base sufficient for multiple-day delivery.

In certain embodiments, the method comprises including an in-line rate-control adhesive more proximal to the user's body surface relative to the reservoir layer.

In certain embodiments, the patch size is from 4 cm² to 40 cm² including polyisobutylene (PIB) as the in-line rate-control adhesive such that the PIB controls the delivery rate of apixaban free base at 4 to 12 mg per day.

In certain embodiments, the method comprises dissolving or dispersing the apixaban free base in a hot melt of poly(ethylene-co-vinyl acetate) (EVA) for the reservoir layer, further comprising disposing a tie layer between the reservoir layer and in-line rate-control adhesive.

In certain embodiments, the method comprises dissolving or dispersing the apixaban free base in a hot melt of EVA for the reservoir layer, wherein the backing layer contains EVA, further comprising disposing an EVA tie layer between the reservoir layer and the in-line rate-control adhesive that comprises PIB.

In certain embodiments, the backing layer has EVA and the tie layer EVA and the backing layer EVA both have lower vinyl acetate content than the EVA in the reservoir layer.

In certain embodiments, the backing layer has EVA and the tie layer EVA and the backing layer EVA both have lower vinyl acetate content than the EVA in the reservoir layer, the reservoir layer EVA having 20-50 wt % vinyl acetate.

In certain embodiments, the method comprises including apixaban free base in the reservoir layer adequate for delivery for 1 to 7 days.

In certain embodiments, the method comprises including in the reservoir layer 20-50 wt % apixaban free base and 60-80 wt % EVA.

In certain embodiments, the reservoir layer is substantially free of an adhesive polymer with acid functionality.

Provided herein is a method of treating thrombosis or related disorder in a subject comprising administering a transdermal delivery patch, wherein the transdermal delivery patch comprising: (a) backing layer; and (b) a matrix layer or a reservoir layer disposed proximally relative to the backing layer, said matrix layer or reservoir layer comprising a polymeric composition containing an amount of apixaban free base sufficient for multiple-day delivery.

In certain embodiments, the transdermal patch is applied to the human subject for a period of about 24 hours.

In certain embodiments, about 1 mg to about 20 mg of apixaban is delivered from the transdermal patch to the human subject daily.

In certain embodiments, the apixaban or a pharmaceutically acceptable salt thereof is in an amount ranging from about 5% to about 10% by weight (wt %) relative to total weight of the drug-containing layer, or the reservoir layer.

In certain embodiments, the adhesive is Duro-Tak™ 87-235A (DT 235A), Duro-Tak™ 87-2054 (DT 2054), Duro-Tak™ 87-2353 (DT 2353), or combinations thereof. In certain embodiments, the hydroxyl functional group containing acrylic-based polymer is Duro-Tak™ 87-2516 (DT 2516), Duro-Tak™ 87-2510 (DT 2510), or a combination thereof. In certain embodiments, the drug-containing layer, or the reservoir layer of the present dermal patch (or the present composition) comprises DT 235A and DT 2516. In certain embodiments, the drug-containing layer, or the reservoir layer of the present dermal patch (or the present composition) comprises DT 2054 and DT 2510. In certain embodiments, the acrylic-based polymer is DT 2287, DT4287, DT788, DT2052, DT2054, DT2353, DT2196, DT2852, DT2074, DT900A, DT9301, DT4098, DT9088, or a combination thereof.

In certain embodiments, the adhesive is obtained from a polymer solution of cross-linked acrylates copolymer comprising acrylic acid and 2-ethylhexyl acrylate. In certain embodiments, the hydroxyl group-containing acrylic-based polymer is obtained from a polymer solution of acrylates copolymer comprising 2-hydroxyethyl acrylate or from a polymer solution of acrylates copolymer comprising vinyl acetate and 2-hydroxyethyl acrylate.

In certain embodiments, the adhesive is in an amount ranging from about 20% to about 25% by weight (wt %), from about 25% to about 30% by weight (wt %), from about 30% to about 35% by weight (wt %), from about 35% to about 40% by weight (wt %), from about 40% to about 45% by weight (wt %), from about 45% to about 50% by weight (wt %), from about 50% to about 55% by weight (wt %), from about 55% to about 60% by weight (wt %), from about 60% to about 65% by weight (wt %), from about 65% to about 70% by weight (wt %), from about 70% to about 75% by weight (wt %), or from about 75% to about 80% by weight (wt %), from about 80% to about 85% by weight (wt %), from about 85% to about 90% by weight (wt %), from about 90% to about 95% by weight (wt %), or from about 95% to about 99% by weight (wt %), relative to the total weight of the drug-containing layer, or the reservoir layer (or the total weight of the composition).

In certain embodiments, the adhesive provides a solubility for apixaban, or a pharmaceutically acceptable salt thereof, of about 1% to about 5%, about 5% to about 10%, about 10% to about 15%.

In certain embodiments, apixaban or its pharmaceutically acceptable salt thereof is in an amount ranging from about 0.1% to about 0.5% by weight (wt %), from about 0.5% to about 1% by weight (wt %), from about 1% to about 2% by weight (wt %), from about 2% to about 3% by weight (wt %), from about 3% to about 4% by weight (wt %), from about 4% to about 5% by weight (wt %), from about 5% to about 6% by weight (wt %), from about 6% to about 7% by weight (wt %), from about 7% to about 8% by weight (wt %), from about 8% to about 9% by weight (wt %), from about 9% to about 10% by weight (wt %), from about 10% to about 11% by weight (wt %), from about 11% to about 12% by weight (wt %), from about 12% to about 13% by weight (wt %), about 13% to about 14% by weight (wt %), about 14% to 15% by weight (wt %), about 15% to about 16% by weight (wt %), about 16% to 17% by weight (wt %), about 17% to 18% by weight (wt %), or about 18% to 19% by weight (wt %), or about 19% to 20% by weight (wt %), relative to the total weight of the drug-containing layer, or the reservoir layer (or the total weight of the composition).

The apixaban or salt thereof may be present in the pharmaceutical composition in combination with another active pharmaceutical ingredient. Suitable active pharmaceutical ingredients for combination with apixaban would be known to those of skill in the art.

### 5.2 PERMEATION ENHANCERS

In certain embodiments, the drug-containing layer, or the reservoir layer of the present dermal patch (or the present composition) further comprises a permeation enhancer. In certain embodiments, the permeation enhancer is an alcohol, a fatty acid, a fatty alcohol, a pharmaceutically acceptable solvent, a pharmaceutically acceptable surfactant, or combinations thereof. In certain embodiments, the permeation enhancer is 1,2-propyleneglycol, a polysorbate (e.g., polysorbate 80 or Tween 80), hydroxypropyl cellulose (HPC), or combinations thereof.

In certain embodiments, the drug-containing layer, or the reservoir layer of the present transdermal patch (or the present composition) further comprises one or more permeation enhancers. In certain embodiments, permeation enhancers can affect the lag time and/or the flux rate of the transdermal patch or composition.

Suitable enhancer compositions may include, but is not limited to, aliphatic alcohols, including, but not limited to, saturated or unsaturated higher alcohols having 12 to 22 carbon atoms, such as oleyl alcohol and lauryl alcohol; saturated or unsaturated fatty acid having a chain of 8 to 20 carbons, such as but not limited to linoleic acid, oleic acid, linolenic acid, stearic acid, isostearic acid and palmitic acid; fatty acid esters, such as but not limited to isopropyl myristate, diisopropyl adipate and isopropyl palmitate; alcohol amines, such as but not limited to triethanolamine, triethanolamine hydrochloride and diisopropanolamine; polyhydric alcohol alkyl ethers, such as but not limited to alkyl ethers of polyhydric alcohols such as glycerol, ethylene glycol, propylene glycol, 1,3-butylene glycol, diglycerol, polyglycerol, diethylene glycol, polyethylene glycol, dipropylene glycol, polypropylene glycol, sorbitan, sorbitol, isosorbide, methyl glucoside, oligosaccharides and reducing oligosaccharides, where the number of carbon atoms of the alkyl group moiety in the polyhydric alcohol alkyl ethers is preferably 6 to 20; polyoxyethylene alkyl ethers, such as but not limited to polyoxyethylene alkyl ethers in which the number of carbon atoms of the alkyl group moiety is 6 to 20, and the number of repeating units (e.g. —OCH₂CH₂—) of the polyoxyethylene chain is 1 to 9, such as but not limited to diethylene glycol monoethyl ether, polyoxyethylene lauryl ether, polyoxyethylene cetyl ether, polyoxyethylene stearyl ether and polyoxyethylene oleyl ether; glycerides (i.e., fatty acid esters of glycerol), such as but not limited to glycerol esters of fatty acids having 6 to 18 carbon atoms, where the glycerides may be monoglycerides (i.e., a glycerol molecule covalently bonded to one fatty acid chain through an ester linkage), diglycerides (i.e., a glycerol molecule covalently bonded to two fatty acid chains through ester linkages), triglycerides (i.e., a glycerol molecule covalently bonded to three fatty acid chains through ester linkages), or combinations thereof, where the fatty acid components forming the glycerides include, but are not limited to octanoic acid, decanoic acid, dodecanoic acid, tetradecanoic acid, hexadecanoic acid, octadecanoic acid (i.e., stearic acid) and oleic acid; middle-chain fatty acid esters of polyhydric alcohols with aliphatic tails of 6-20 carbon atoms; alkyl esters such as but not limited to lactic acid alkyl esters and dibasic acid alkyl esters with chain of 1 to 6 carbon atoms; acylated amino acids; pyrrolidone; pyrrolidone derivatives; and combinations thereof.

In certain embodiments, suitable enhancer compositions include, but are not limited to, ethoxylated fatty alcohols, such as but not limited to polyethylene glycol ethers, polyoxyethers of lauryl alcohol, polyethylene glycol ether of cetyl alcohol, polyethylene glycol ethers of stearic acid, polyethylene glycol ethers of oleyl alcohol, polyoxyethylene ethers of a mixture of cetyl alcohol and stearyl alcohol, ethoxylated linear alcohol, and combinations thereof.

In certain embodiments, suitable enhancers include, but are not limited to, lactic acid, tartaric acid, 1,2,6-hexanetriol, benzyl alcohol, lanoline, potassium hydroxide (KOH), and tris(hydroxymethyl)aminomethane. Other suitable permeation enhancers may comprise glycerol monooleate (GMO) and sorbitan monolaurate (SML), lactate esters such as lauryl lactate, methyl laurate, caproyl lactic acid, lauramide diethanolamine (LDEA), dimethyl lauramide, polyethylene glycol-4 lauryl ether (Laureth-4), lauryl pyroglutamate (LP), sorbitan monolaurate, ethanol and combinations thereof.

Permeation enhancers may also comprise surfactants including combinations of semi-polar solvents, e.g., propylene glycol, butane diol, N-methylpyrrolidone, dimethyl sulfoxide, diethylene glycol methyl ether and dimethyl isosorbide. Other surfactant permeation enhancers may comprise isopropyl myristate, oleic acid, lauryl lactate and combinations thereof. Furthermore, in certain embodiments, permeation enhancer may comprise squalane, isopropyl palmitate, isopropyl myristate, sorbitan laurate, DL-limonene, ethyl oleate, methyl dodecanoate, propylene glycol dicaprylocaprate, propylene glycol dicaprylate/dicaprate, Labrafac™ PG, octyl alcohol, dodecyl alcohol, polyoxyethylene (4) lauryl ether, Brij® 30, oleyl alcohol, polyoxyethylene sorbitan monooleate, Tween®80, propylene glycol, diethylene glycol, monoethyl ether, propylene glycol monocaprylate, Capryol PGMC, 1-methyl-2-pyrrolidinone, glyceryl triacetate, triacetin, polyoxyl castor oil, Kolliphor®RH40, oleoyl macrogol-6 glycerides, Labrafil™ M1944CS, linoleoyl polyoxyl-6 glycerides, Labrafil™ M2125CS, caprylocaproyl macrogol-8 glycerides, labrasol®, polyoxyl castor oil, oleoyl macrogol-6 glycerides, linoleoyl polyoxyl-6 glycerides, caprylocaproyl macrogol-8 glycerides and N-methyl pyrrolidone.

Numerous permeation enhancers were evaluated to identify suitable permeation enhancers, including permeation enhancers listed in Table 2 below that lists solubility of apixaban in the respective permeation enhancers. Preferred permeation enhancers that provide good solubility include organic solvents such as dichloromethane (DCM), as well as 1,3-Dimethyl-2-imidazolidinone (DMI). Apixaban has low solubility in propylene glycol (PG), Tween 80 and other enhancers. Although apixaban has better solubility in fatty acid, it is not stable in fatty acid.

In certain embodiments, the drug-containing layer, or the reservoir layer of the present transdermal patch (or the present composition) comprises one or more enhancers/stabilizers selected from the group consisting of: Kollisolv PG, hydroxypropyl cellulose (HPC), and Tween 80.

In some embodiments, the drug-containing layer, or the reservoir layer of the present transdermal patch (or the present composition) comprises a combination of two or more permeation enhancers. In certain embodiments, the drug-containing layer, or the reservoir layer of the present transdermal patch (or the present composition) comprises a combination of aliphatic alcohols, fatty acids, fatty acid esters, alcohol amines, polyhydric alcohol alkyl ethers, polyoxyethylene alkyl ethers, glycerides, middle-chain fatty acid esters of polyhydric alcohols, lactic acid alkyl esters, dibasic acid alkyl esters, acylated amino acids, pyrrolidone, pyrrolidone derivatives, ethoxylated fatty alcohols and/or surfactants. In another embodiment, the apixaban transdermal patch of the present invention comprises a combination of fatty acids and/or fatty alcohols, such as oleic acid and lauric acid, oleic acid and lauryl alcohol, oleyl alcohol and lauric acid or oleyl alcohol, lauryl alcohol, surfactants or a combination thereof.

In some embodiments, the permeation enhancers comprise aliphatic alcohols, fatty acids, fatty acid esters, alcohol amines, polyhydric alcohol alkyl ethers, polyoxyethylene alkyl ethers, glycerides, ethoxylated fatty alcohols, or a combination thereof. In other embodiments, the permeation enhancers comprise methyl laurate, proptlene glycol, transcutol P, brij 30, ethyl oleate, oleic acid, isopropyl myristate, lauryl alcohol, surfactants or a combination thereof.

In certain embodiments, the drug-containing layer, or the reservoir layer of the present transdermal patch (or the present composition) comprises a combination of permeation enhancers with apixaban solubility about 5 mg/mL to about 10 mg/mL, about 10 mg/mL to about 15 mg/mL, about 15 mg/mL to about 20 mg/mL, about 20 mg/mL to about 25 mg/mL, about 25 mg/mL to about 30 mg/mL, or about 30 mg/mL to about 35 mg/mL.

In some embodiments, the present transdermal patch of the present invention comprises a combination of methyl laurate and propylene glycol permeation enhancers wherein the weight ratio of the content of methyl laurate to the content of propylene glycol is about 2:1.

In some embodiments, the present transdermal patch comprises a combination of Transcutol P and Brij 30 wherein the weight ratio of the content of Transcutol P to the content of Brij 30 is preferably about 1:1.

In some embodiments, the present transdermal patch comprises a combination of ethyl oleate and oleic acid wherein weight ratio of the content of ethyl oleate to the content of oleic acid is preferably about 1:1.

In some embodiments, the present transdermal patch comprises a combination of isopropyl myristate and lauryl alcohol. In certain embodiments, the weight ratio of isopropyl myristate to lauryl alcohol is about 1:1.

In some embodiments, the present transdermal patch comprises a combination of propylene glycol, Transcutol P and Brij 30. In certain embodiments, the weight ratio of propylene glycol to Transcutol P to Brij 30 is about 1:1:1.

In certain embodiments, the permeation enhancer (or a combination of permeation enhancers) is in an amount ranging from about 0.5% to about 1% by weight (wt %), from about 1% to about 2% by weight (wt %), from about 2% to about 5% by weight (wt %), from about 15% to about 20% by weight (wt %), from about 20% to about 25% by weight (wt %), from about 25% to about 30% by weight (wt %), from about 30% to about 35% by weight (wt %), from about 35% to about 40% by weight (wt %), from about 40% to about 45% by weight (wt %), from about 45% to about 50% by weight (wt %), from about 50% to about 55% by weight (wt %), or from about 55% to about 60% by weight (wt %), relative to the total weight of the drug-containing layer, or the reservoir layer (or the total weight of the composition). In some embodiments, the drug-containing layer, or the reservoir layer of the present dermal patch (or the present composition) comprises two or more permeation enhancers in a total amount of about 10% of the drug-containing layer, or the reservoir layer of the present dermal patch (or the present composition). In some embodiments, the present transdermal patch comprises two or more permeation enhancers in a total amount of about 15% of the drug-containing layer, or the reservoir layer of the present dermal patch (or the present composition). In some embodiments, the present transdermal patch comprises two or more permeation enhancers in a total amount of about 20% of the drug-containing layer, or the reservoir layer of the present dermal patch (or the present composition).

### 5.3 SOLVENTS

In certain embodiments, the drug-containing layer, or the reservoir layer of the present dermal patch (or the present composition) further comprises an organic solvent. In certain embodiments, the organic solvent is 1,3-Dimethyl-2-imidazolidinone (DMI), dichloromethane (DCM), methanol, ethanol, ethyl acetate, methyl ethyl ketone (MEK), cyclohexane, isopropanol, acetyl acetone, toluene, xylene, 2, 4-pentanedione, n-heptane, heptane, chloroform, tetrahydrofuran (THF), acetone, propanol, 1-propanol, 2-propanol, methyl acetate, isopropyl acetate, butyl acetate, 2-methyl-1-propanol, or a combination thereof. In certain embodiments, the organic solvent is DMI, DCM, ethyl acetate, heptane, n-heptane, hexane methanol, ethanol, isopropanol, 2,4-pentanedione, toluene, xylene, or combinations thereof.

In certain embodiments, the organic solvent is in an amount ranging from about 0.5% to about 1% by weight (wt %), from about 1% to about 2% by weight (wt %), from about 2% to about 5% by weight (wt %), from about 5% to about 10% by weight (wt %), from about 10% to about 15% by weight (wt %), from about 15% to about 20% by weight (wt %), from about 20% to about 25% by weight (wt %), from about 25% to about 30% by weight (wt %), from about 30% to about 35% by weight (wt %), from about 35% to about 40% by weight (wt %), from about 40% to about 45% by weight (wt %), or from about 45% to about 50% by weight (wt %), relative to the total weight of the drug-containing layer (or the total weight of the composition).

In certain embodiments, the drug-containing layer, or the reservoir layer of the present dermal patch (or the present composition) further comprises a crystallization inhibitor. Such crystallization inhibitor includes but are not limited to HPC, HEC, HPMC, PEG (different molecular weight), PVP/VA Copolymer, copolymers of methacrylic acid i.e. (Eudragit® E, Eudragit® L, Eudragit® RL, Eudragit® S, Eudragit® RS,), polyvinylpyrrolidone (PVP) and its derivatives; dextrin derivatives; polyethylene glycol (PEG); polypropylene glycol (PPG), polyvinyl alcohol (PVA), poloxamers.

In certain embodiments, the drug-containing layer, or the reservoir layer of the present dermal patch (or the present composition) further comprises an antioxidant. For example, the pharmaceutically acceptable antioxidant may be selected from the group consisting of ascorbic acid, sodium ascorbate, sodium bisulfate, sodium metabisulfate and monothio glycerol. α-Tocopherol, Gamma-tocopherol, Delta-tocopherol, Vitamin E, Butylated hydroxytoluene (BHT), Butylated hydroxyanisole (BHA), Tertiary-butyl hydroquinone (TBHQ), Propyl gallate, Octyl gallate, Dodecyl gallate, Sodium erythorbate, Erythorbic Acid, 4-Hexylresorcinl, Calcium ascorbate, Fatty acid esters of ascorbic acid (ascorbyl palmitate), or a combination thereof.

### 5.3 FLUX RATE

In certain embodiments, the present transdermal patch is a daily apixaban transdermal patch, which provides a steady state flux rate at about 0.5 μg/cm²·hr and up to about 20 μg/cm²·hr as well as a lag time of less than about 8 hours.

In certain embodiments, the present transdermal patch may be about 150 cm² or less, about 120 cm² or less, about 100 cm² or less, about 80 cm² or less, about 60 cm² or less, about 40 cm² or less, about 5 cm² to about 120 cm², about 40 cm² to about 100 cm², or about 60 cm² to about 80 cm².

In certain embodiments, the apixaban transdermal patch of the present invention comprises a drug-containing layer, or the reservoir layer that comprises apixaban free base or a pharmaceutically acceptable salt thereof as the active pharmaceutical ingredient.

The amount of apixaban (free base or salt) dissolved in the drug reservoir layer matrix (on a solid, or dry, basis) can be about 15 wt % to 45 wt %, preferably about 20 wt % to 40 wt %, preferably above 25 wt %, more preferably from about 25 wt % to 40 wt %, even more preferably about 25 wt % to 35 wt %. The balance of the material in the reservoir layer can be the carrier material. Optionally, other excipients can be included. Such apixaban contents are, for example, suitable for patches of about 10 to 40 cm² with thickness of about 1 mil (0.025 mm) to 12mil (0.3 mm) and having a dose of about 2.5 mg to 10 mg. Such apixaban contents are suitable for effecting flux of therapeutic effect for ailments such as thrombosis, etc., with a flux (in microgram (meg or μg) per unit area time) of, e.g., greater than 1 mcg/(cm² hr), preferably greater than about 2 mcg/(cm² hr), more preferably about 3 mcg/(cm² hr) to 80 mcg/(cm² hr), more preferably about 4 mcg/(cm² hr) to 50 mcg/(cm² hr), more preferably about 4 mcg/(cm² hr) to 25 mcg/(cm² hr) for a 1 -day patch or multiple-day patch (e.g., a 3-day patch, 7-day patch). For a patch for one-day use, the concentration and size can be on the lower end of the ranges, e.g., the apixaban content can be about 10 to 20 wt %, a size of about 5 to 20 cm² with thickness of about 1 mil (0.024 mm) to 4mil (0.1 mm). Conversely, for a three or more day patch, the drug content and size can be in the larger end of the ranges, e.g., the apixaban content can be about 20 to 45 wt %, a size of about 10 to 40 cm²with thickness of about 1 mil (0.024 mm) to 12mil (0.1 mm), although a size of 20 cm² or less with thickness 4 mil or less can also be used.

### 5.4 TRANSDERMAL PATCH WITH RESERVOIR

In one embodiment, the transdermal delivery system comprises a backing layer, a drug reservoir layer located on the skin (body surface) side of the backing layer, a body-contacting adhesive on the body side of the drug reservoir layer, and a peelable protective layer (or release liner) further on the body side of the body-contacting adhesive. Upon use, the protective layer or release liner is removed and the device is applied such that the body-contacting adhesive is applied to contact the body surface (e.g., skin). The body-contacting adhesive adheres securely to the body surface. The body-contacting adhesive can also contain the drug and permeation enhancer, as well as other ingredients. The reservoir layer is a matrix of carrier material that is suitable for carrying the pharmaceutical agent (or drug) apixaban for transdermal delivery. Preferably, the whole matrix, with drugs and other optional ingredients, is a material that has the desired adhesive properties. The polymer that makes up the matrix in the reservoir layer provides the structure for carrying the drug (and other excipients that optionally may be present). However, even if the reservoir layer matrix does not have adequate adhesive property to adhere directly to the body surface, the body-contacting adhesive will have adhesive property to retain the drug delivery device on the body surface (e.g., skin) for the period desired, whether one day, three days, or seven days. Although the drug and other ingredients carried in the matrix can be above saturation in a multiple phase polymeric composition, preferably at least the drug, and preferably all the other ingredients carried by the matrix in the reservoir layer are in a single phase polymeric composition in that no drug is undissolved. In certain embodiments, all other components are present at concentrations no greater than, and in certain embodiments, less than, their saturation concentrations in the reservoir layer, without undissolved material. In certain embodiments, it is a composition in which all components are dissolved. The reservoir layer can be formed using a pharmaceutically acceptable polymeric material that can be an acceptable adhesive for application to the body surface. In one embodiment, the body-contacting adhesive provides good adhesive property to ensure that the device stays attached to the body surface over the desired period. In a multiple phase polymeric composition, at least one component, for example, a therapeutic drug, is present in amount more than the saturation concentration and some of the drug may be in undissolved form, e.g., crystals or particulates. In some embodiments, more than one component, e.g., a drug and a permeation enhancer, is present in amounts above saturation concentration. In certain embodiments, the adhesive acts as the reservoir layer and includes a drug, such as apixaban.

In one embodiment, the body-facing surface of the reservoir layer may be formulated with a thin adhesive coating. The reservoir layer may be a single phase polymeric composition or a multiple phase polymeric composition.

With the use of an overlay adhesive, a skin-contacting adhesive may not be needed. Further, in certain cases, a rate-control layer can also be positioned on the reservoir layer proximal to the skin. Any suitable rate-control material known in the art can be used.

The reservoir layer may be formed from drug (or biological active agent) reservoir layer materials suitable for delivery of apixaban or its salts. For example, the drug reservoir layer is formed from a polymeric material in which the drug can be included, all in dissolved form, for the drug to be delivered within the desired range, such as, a polyurethane, ethylene/vinyl acetate copolymer (EVA), polyacrylate, styrenic block copolymer, gel polymer, and the like. Although it may be possible to blend different matrix polymers, in certain embodiments, one matrix polymer is the main component of the matrix layer (i.e., more than 50 wt %, preferably more than about 90 wt %, and substantially all, and even more preferably all of the matrix carrier material) in the matrix polymeric material. In certain embodiments, the reservoir layer is formed from a pharmaceutically acceptable EVA. The drug reservoir layer or the matrix layer can have a thickness of about 1-20 mils, about 1-10mils (0.025-0.25 mm), about 2 mils to 5 mils (0.05 mm to 0.12 mm), about 2 mils to 3 mils (0.05 mm to 0.075 mm).

In one embodiment, the reservoir layer is a monolithic polymeric adhesive layer that contains the drug and also provides adhesion for attaching to the body surface such that the device consists of only the three layers. In one embodiment, an overlay is positioned on top (at a position most distal from the body surface) of the device. The overlay has an adhesive on its body surface facing side to attach to the body surface. In certain embodiments, a rate-controlling layer is positioned adjacent or next to the reservoir layer proximal to the skin. Any suitable rate-controlling material described herein or known in the art can be used.

In certain embodiments, the drug-containing layer, or the reservoir layer of the present transdermal patch further comprises one or more polymers for housing the active agent (e.g., apixaban or a pharmaceutically acceptable salt thereof) that play a significant role in determining apixaban flux rate. Specifically, higher flux rate may be achieved by lowering the solubility of the apixaban within the polymer(s) relative to the solubility within the stratum corenum layer of the user's skin. However, low solubility of apixaban may cause crystallization of apixaban within the skin patch, reducing the amount of apixaban available to be delivered to a user. In addition, low solubility of respective ingredients of the transdermal patch, or low miscibility, could present manufacturing issues as it could prevent even distribution of apixaban within the polymers and cause phase separation. Therefore, solubility of apixaban within the polymers and miscibility of respective components of the transdermal patch are important considerations that necessitate proper balancing when selecting polymers and creating formulations using the selected polymers for the transdermal patch of the present invention.

In one embodiment, EVA copolymer is a used as the matrix carrier for carrying apixaban or its salts and optionally other ingredients in the reservoir layer. EVA copolymers are thermoplastic hot-melt adhesives. They are typically manufactured in high pressure copolymerization processes. Hot melt thermoplastic material provides an advantage in that little or no solvent, especially organic solvent, need to be used to make a flowable casting material to make a layer of apixaban containing matrix. With a hot melt material, the apixaban can be dispersed evenly in the hot melt adhesive, or it can be completely dissolved therein without the presence of crystalline or particulate apixaban or its salts. EVA copolymers are conventionally considered to be copolymers of ethylene and vinyl acetate in which generally the weight percentage of ethylene in the polymer molecule is more than that of the vinyl acetate. In certain embodiments, the vinyl acetate content is 5 wt %-10 wt %, 10 wt %-20 wt %, 20-40 wt %, 40-50 wt %, 50-70 wt %, 70-80 wt %. Generally, the vinyl acetate content is about 4 wt % to 50 wt %, about l0 wt % to 49 wt %. For use as the carrier material in the apixaban reservoir layer, the vinyl acetate content is about 10-20 wt %, 20-35 wt %, 35-45 wt %, 45-60 wt %, 60-70 wt % vinyl acetate. In certain embodiment, EVA is about 10-30 wt %, 30-40 wt % and 0.5 wt %-1.5 wt %, 1.5-2 wt % vinyl acetate. The higher the ethylene content, the more compatibility and adhesion the EVA has with nonpolar material such as polyolefins. In certain embodiments, the transdermal system comprises EVA of about 15-30 wt % or 30-35 wt % and about 35 wt % to 45 wt % vinyl acetate, for forming the drug reservoir layer for delivery of apixaban at a desirable flux and for a suitable period of delivery. Generally, the EVA number represents the percent vinyl acetate in the EVA polymer, thus EVA40 has 40 wt % vinyl acetate and EVA20 has 20 wt % vinyl acetate, etc. The EVA polymer may optionally be modified by methods well known in the art, including modification with an unsaturated carboxylic acid or its derivatives, such as maleic anhydride or maleic acid.

EVA materials are commercially available from various suppliers, e.g., Minnesota Mining Co and DuPont (e.g., EL V AX ®). Methods for their preparation are, for example, described in U.S. Pat. Nos. 2,200,429 and 2,396,785. For the EVA of the present invention, EVA copolymers having vinyl acetate content of about 2-4%, 4-20%, 20-40%, 40-60%, 60-80%, 80-90%% by weight of the total and a melt index of about 0.1 to 1000 grams per ten minutes can be used. Melt index is the number of grams of polymer that can be forced through a standard cylindrical orifice under a standard pressure at a standard temperature and thus is inversely related to a molecular weight, as determined by standard ASTM D 1238-65T condition E practice. The melt index for EVA for the reservoir layer is from about 0.3-3, 3-20, 20-50, 50-60, 60-80, 80-100.

The device can include an in-line adhesive at a position more proximal to the body surface than the apixaban-containing reservoir layer. Further, the in-line adhesive can be put in for rate-controlling function to reduce the flux through the body surface. For example, the in-line adhesive can be the body-contacting adhesive layer that is disposed on the body-facing side of the apixaban-containing reservoir layer. In certain embodiments, more layers can be disposed on the body proximal side of the apixaban-containing reservoir layer either before a rate-control layer or after the rate-control layer. In certain embodiments, the rate-control layer is the in-line body-contacting adhesive. Such a structure will facilitate the ease of making of the device, because fewer layers are included. The rate-control adhesive slows the flux of apixaban to a level that is suitable to deliver the drug at a therapeutically effective rate of greater than about 1-3 mcg/(cm² hr); about 3-5 mcg/(cm² hr), about 5-8 mcg/(cm²hr), about 10-30 mcg/(cm²hr), about 30-80 mcg/(cm²hr), about 8 mcg/(cm² hr) to 60 mcg/(cm² hr). Without the rate-control adhesive, the flux would have been higher, unless another rate limiting layer is used.

In one embodiment, the in-line, rate-control adhesive is made of a material that is different from the apixaban-containing layer. In one embodiment, the rate-control adhesive is made of polyisobutylene (PIB). PIB has excellent adhesive property and is suitable for retaining the device on body surface for 1-day delivery or multiple day delivery, i.e., 2-day, 3-day, etc., even up to 7-day delivery. PIB adhesives are mixtures of high molecular weight (HMW) PIB, low molecular weight (LMW) PIB, and/or plasticizer such as polybutene. Such mixtures are described in the art, e.g., U.S. Pat. No. 5,508,038. The molecular weight of the HMW PIB is usually in the range of about 700,000 to 2,500,000 Da, whereas that of the LMW PIB typically ranges from about 1,000 to about 90,000, about from 35,000 to 50,000. The molecular weights referred to herein are weight average molecular weights. The weight ratio of HMW PIB to LMW PIB in the adhesive ranges between about 1:1 to 1:20, preferably about 1:3 to 1:10. By adjusting the ratio of HMW and LMW PIB or using plasticizer, the rheological properties of the PIB adhesive can be tailored so that the desired adhesive properties can be achieved. Generally, higher amount of LMW PIB and the use of plasticizer will decrease modulus but increase cold flow.

In certain embodiments, the adhesive composition contains the HMW and LMW PIB in weight ratios (HMW PIB:LMW PIB) in the range of about 3-40:97-60, in the range of about 5-25:95-75 and in the range of about 10-20:90-80. The ratio of HMW PIB to LMW PIB that provides an optimal adhesive for a specific drug agent will be dependent upon the identity and concentration of agent being delivered. As an example, in one effective embodiment the PIB adhesive includes 5 wt % HMW PIB material (such as OPPANOL L80, L1OO, and L 140 from BASF) and 95 wt % LMW PIB material (Such as OPPANOL B10, B 11, B12, and B13 from BASF). Such an exemplary PIB adhesive 2-3 mil (0.05 mm to 0.075 mm) in thickness demonstrated rate-control when combined with a apixaban (35 wt %) in EVA40 (ethylene -vinyl acetate copolymer with 40% vinyl acetate, such as EL V AX ® 4 OW from DuPont) drug reservoir layer about 4-7 mil (0.1 to 0.175 mm) thick, resulting in apixaban base average flux of (5.5 μg/cm²-h).

Optionally, modification of flux of the drug through the PIB can be effected by incorporating in the adhesive material such as micronized, crosslinked polyvinylpyrrolidone (PVP), such as CROSPOVIDONE (Kollidon CL-CY from BASF typically with bulk density between 0.2-0.3 g/cm3 and particle size D90 of 10-20 micron). Such PVP improves the permeability of apixaban material through the PIB layer. For example the 5:95 L100:B12 PIB adhesive was formulated to include 20 wt % CROSPOVIDONE, from which an average apixaban base flux of 33.3 μg/(cm²-h) was achieved.

Varying the amounts of CROSPOVIDONE in the PIB adhesive would result in fine-tuning the flux of apixaban to desired levels. For example, multilaminate formulations containing different PVP amounts were tested for the effect of such variation on flux through skin. The multilaminate formulations contained 35 wt % of apixaban base in EVA40, an EVA12 as tie layer and PIB adhesive (5:95 L100:B12 PIB) with 12 wt % or 18 wt % PVP were tested. The formulation containing 12 wt % PVP resulted in a flux of 5.4 μg/(cm² h) whereas that from the formulation containing 18 wt % PVP resulted in a flux of 7.5 μg/(cm² h). A formulation containing 30 wt % apixaban base and 15 wt % PVP was also tested. The flux of apixaban formulation from this formulation was 6.3 μg/(cm² h). This experiment indicated that the amount of PVP added in the PIB adhesive could be adjusted to change the flux of apixaban base to desired levels. PIB polymers are available commercially, e.g., under the tradename VISTANEX™ from Exxon Chemical. The amount of PVP in the resulting adhesive can be about lwt % to 30 wt %, preferably about 5 wt % to 25 wt %, more preferably about 8 wt % to 20 wt %.

The term, “plasticizer” as used herein relating to PIB refers to compounds other than the agent being delivered, such as mineral oil, polybutene oil, and other low molecular weight hydrocarbons that act to plasticize PIB adhesives and increase their permeability to the agent being delivered. An adhesive composition is substantially free of plasticizer if it contains, at most, trace amounts of plasticizer and more preferably, no plasticizer. The term, “tackifier” as used herein relating to PIB refers to material, other than PIB, that is added to adhesives to increase their tack or stickiness. Such materials are typically naturally occurring resinous or resinous materials or synthetic polymer materials. An adhesive is substantially free of tackifier if it contains, at most, trace amounts of tackifier and preferably no tackifier.

The PIB can be with or without tackifiers or plasticizers, such as low molecular weight polybutene (e.g., INDOPOL H 1900 and/or high Tg, low molecular weight aliphatic resins such as the ESCOREZ resins available from Exxon Chemical, and the like). The body-contacting adhesive can be further modified to improve body surface adhesion. For example, a body-contacting adhesive containing 16 wt % L1OO PIB, 24 wt % OPPANOL B12 PIB, 40 wt % INDOPOL H1900 polybutene, and 20 wt % CROSPOVIDONE will provide superior body surface adhesion and continue to provide rate-control. Rate-control can be further adjusted by adjusting the body-contacting adhesive thickness, for example, by increasing the adhesive thickness to provide greater rate-control.

The thickness of the in-line adhesive layer (which optionally can also be rate-controlling) will generally be from about 0.5 mil (0.127 mm) to 6 mil (0.154 mm), preferably about 2 mil (0.05 1 mm) to 3 mil (0.076 mm). Although it is desired that the adhesive functions in rate-control, the composition and thickness of the adhesive layer is provided such that the adhesive layer does not constitute a significant permeation barrier to the passage of the agent to be delivered but act adequately for rate-controlling function if rate-control is desired in the adhesive. PIB is particularly useful in this respect. Unless a drug requires the use of a loading dose to rapidly saturate drug delivery sites in the skin, the adhesive thickness is also preferably selected so that the adhesive does not contain a substantial amount of drug agent and preferably less than about 15 wt % of the total amount of the drug agent in the patch.

Yet another adhesive that can be used for in-line adhesive, which can be a body-contacting adhesive, is a polyacrylate (acrylic polymers), e.g., polyacrylate described in US patent publication US20040213832. A preferred type of polyacrylate is made from monomeric esters, preferably monomeric esters of alcohols that have 1 to 8 carbon atoms in the alcohol. Preferred alcohols include alkyl alcohol, hydroxyalkyl alcohol, methoxyalkyl alcohol and vinyl alcohol. A preferred monomeric ester has only one such 1 to 8 carbon atoms group from an alcohol and one organic group from an organic acid (e.g., acrylic acid and methacrylic acid). Examples of polyacrylate -based adhesives are as follows, identified as product numbers, manufactured by National Starch (Product Bulletin, 2000, DURO-TAK® is a trademark of National Starch adhesives): 87-4098, 87-2287, 87-4287, 87-2516, 87-2051, 87-2052, 87-2054, 87-2196, 87-9259, 87-9261, 87-2979, 87-2510, 87-2353, 87-2100, 87-2852, 87-2074, 87-2258, 87-9085, 87-9301 and 87-5298. DURO-TAK ® 87-2287 and 87-4287 both are polymeric adhesives derived from monomer compositions that are similar: 5.2 wt % 2-hydroxy ethyl acrylate, about 20-40 wt % vinyl acetate, and about 55-75 wt % 2-ethylhexyl acrylate; and these two polymeric adhesives are provided solubilized in ethyl acetate in solids content of about 40-50 wt %. The DURO-TAK® 87-4287 monomeric components consist of the above-mentioned three monomeric esters. The DURO-TAK® 87-2287 adhesive is derived from monomeric components consisted of four monomers: vinyl acetate, 28%; 2-ethylhexyl acrylate, 67%; hydroxyethyl acrylate, 4.9%; and glycidyl methacrylate, 0.1%, see U.S. Pat. No. 5,693,335. In certain workable embodiments, the adhesive has little or no acid functionality. Preferably it is substantially free of an adhesive polymer of acrylic acid or (meth) acrylic acid. In such adhesives, there is little or no adhesive that is polymerized from monomeric components of acrylic acid or (meth) acrylic acid. For example, the adhesive can have 4 wt % or less of a polymer that is polymerized from acrylic acid or (meth) acrylic acid monomers. It is preferred that a polyacrylate adhesive be used with a rate-control tie layer such as EV A9 and/or EVA12, and/or EVA 18.

Another kind of in-line body-contacting adhesive that can be used is a silicone adhesive. The silicone adhesives that may be used are typically high molecular weight poly dimethyl siloxanes or polydimethyldiphenyl siloxanes. Formulations of silicone adhesives that are useful in transdermal patches are described in U.S. Pat. Nos. 5,232,702, 4,906,169 and 4,951,622. One example of such a silicone adhesive is Silicone 4202 polydimethylsiloxane adhesive from Dow Corning. It is noted that other polysiloxane pressure sensitive adhesives can be used. Similar to the above in-line adhesives, the thickness can be adjusted by one skilled in the art based on whether the body-contacting adhesive is to have a rate-controlling function, in view of the present disclosure. EVA tie layer(s) can also be used with a silicone in-line body-contacting adhesive.

In certain embodiments, one or more tie layers can be included in the patch. To increase bonding of the EVA apixaban reservoir layer to the body-contacting adhesive (e.g., PIB) for secure attachment such that delamination can be prevented, the apixaban delivery device can include a tie-layer (or multiple layers if desired) of EVA with a vinyl acetate concentration less than that of the EVA in the apixaban reservoir layer, the vinyl acetate concentration being preferably about 8 wt % or more and less than about 40 wt %, preferably about 20 wt % or less, more preferably about 9 wt % to 20 wt %, even more preferably about 9 wt % to 18 wt % (e.g., adhesive EVA12), even more preferably about 9 wt % to 10 wt %. The reduced vinyl acetate content in the tie layer compared to the drug reservoir layer improves the tie layer's compatibility with the nonpolar PIB or other nonpolar or less polar adhesives and results in a stronger bond than if an EVA with a higher vinyl acetate content is used. Such a tie layer was found by peel testing to provide increased bond strength to prevent delamination. It has been demonstrated through in-vitro flux testing that permeation is restricted by the inclusion of a 1 mil (0.025 mm) EV A9 membrane between an apixaban reservoir layer (e.g., of EVA40) and a body-contacting adhesive (e.g., the PIB embodiments described above) more permeable than the EV A9. The use of a tie layer of a 1 mil (0.025 mm) EVA12 membrane or EVA1 8 membrane between a apixaban reservoir layer (e.g., of EVA40) and a highly permeable body-contacting adhesive did not affect the permeation significantly. The thickness of the tie layer is about 0.5 mil (0.0.0127 mm) to 5 mil (0.0625 mm), about 0.5 mil (0.0127 mm) to 2 mil (0.05 mm), about 0.5 mil (0.0127 mm) to 1 mil (0.0254 mm), about 1 mil (0.0254 mm) to 2 mil (0.05 mm). Minimized thickness in the tie layer and selection of a tie layer that has little rate-controlling function is preferred if it is desired to reduce risk of rate-control effect, if any, caused by the tie-layer. In certain embodiments, the material and the thickness of tie layer also contribute to the rate-controlling function, along with the rate-control adhesive (e.g., PIB).

Generally, an EVA tie layer is laminated to an EVA drug reservoir layer by heat pressing so that the tie layer and the drug reservoir layer fuse together. Adhesive is typically heat-cast on a separate carrier liner material. Then the EVA drug reservoir layer laminate and adhesive laminate are laminated together to obtain final product. Typically, the EVA drug reservoir layer is heat-cast on a carrier liner material first for easier processing as a laminate and then the laminate is further laminated with the tie layer by heat.

In certain embodiments, in-line body-contacting adhesive that has less rate-controlling function is used and depends on the rate-control function of the tie layer to control the rate of apixaban delivery. For example, if the skin-contacting adhesive allows higher flux levels, additional rate-control could be added by modifying the tie-layer with reduced vinyl acetate content (such as using an EVA9, having 9 wt % vinyl acetate) to reduce the drug transport rate. Further the tie-layer thickness can also be modified to affect the drug transport rate (thicker to reduce the transport, or thinner to increase the transport).

For forming the reservoir layer, an alternative polymer forms a gel-like reservoir layer (e.g., one with hydrogel polymer). Various drug reservoir layer compositions can be utilized according to this invention include aqueous and non-aqueous drug reservoir layer compositions. A typical general aqueous formulation is shown in Table 1.

### Table 1

Reservoir layer components:
79.4% by weight water,
14.00% by weight of ethanol,
4.7% by weight of apixaban
1.9% by weight of gelling agent (Hydroxy ethyl cellulose)

Solvents used in the aqueous and non-aqueous systems include but are not limited to ethanol, isopropanol, butylene glycol, cremaphor EL, glycerol, isopropyl myristate, isopropyl palmitate, isopropyl stearate, diisopropyl adipate, labrafil, labrasol, oleic acid, mineral oil, myglyol, plurol oleic, propylene carbonate, propylene glycol, polyoxyethylene glycol (PEG), and silicone solvent like cyclomethicone, hexamethyldisiloxane, solutol, sorbitol or transcutol P. For reservoir layer systems containing

gels, the gelling agent can be CARBOSIL polyurethane elastomer, CARBOPOL polyacrylic acid polymer, hydroxyethyl cellulose, hydroxypropyl cellulose, hydroxypropylmethyl cellulose, klucel or other known gelling agents. In an aqueous reservoir layer, the apixaban can be formulated in an aqueous environment. Suitable polymers for the gel matrix can contain essentially any nonionic synthetic and/or naturally occurring polymeric materials. A polar nature is preferred, since apixaban base and its salts are polar, to promote compatibility and enhance agent solubility. Optionally, the gel matrix can be water swellable. Examples of suitable synthetic polymers include, but are not limited to, poly(acrylamide), poly(2-hydroxyethyl acrylate), poly(2-hydroxypropyl acrylate), poly(N-vinyl-2-pyrrolidone), poly(n-methylol acrylamide), poly(diacetone acrylamide), poly(2-hydroxylethyl methacrylate), poly(vinyl alcohol), and poly(allyl alcohol). Hydroxyl functional condensation polymers (i.e., polyesters, polycarbonates, polyurethanes) are also examples of suitable polar synthetic polymers. Polar naturally occurring polymers (or derivatives thereof) suitable for use as the gel matrix are exemplified by cellulose ethers, methyl cellulose ethers, cellulose and hydroxylated cellulose, methyl cellulose and hydroxylated methyl cellulose, gums such as guar, locust, karaya, xanthan, gelatin, and derivatives thereof. Typically, the weight percentage of the matrix polymer used to prepare gel matrices for the reservoir layers of the electrotransport delivery devices, in certain embodiments of the methods of the invention, is about l0 wt % to about 30 wt %, preferably about 15 wt % to about 25 wt %.

An in-line, body-contacting adhesive can be disposed on the body surface proximal side of the hydrogel layer to secure the patch on the skin by adhesion. For example, an EVA tie layer can be placed between the hydrogel reservoir layer and a silicone adhesive to facilitate the adhesion of the nonpolar silicone adhesive to the polar hydrogel. In certain embodiments, an overlay with adhesive can be disposed as the top layer of the device for attaching the device on the skin. By controlling the flux by means of the size, thickness of the reservoir layer and the tie layer, if any, and the drug loading in the reservoir layer, devices can be made such that an in-line adhesive is not needed.

Yet another matrix material for the reservoir layer for holding the drug apixaban or a salt thereof is polyacrylate. The polyacrylate (acrylic polymers) are comprised of a copolymer or terpolymer comprising at least two or more exemplary components selected from the group comprising acrylic acids, alkyl acrylates, methacrylates, copolymerizable secondary monomers or monomers with functional groups. Examples of monomers include, but are not limited to, vinyl acetate, acrylic acid, methacrylic acid, methoxyethyl acrylate, methyl acrylate, ethyl acrylate, butyl acrylate, butyl methacrylate, hexyl acrylate, hexyl methacrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, hydroxyethyl acrylate, hydroxypropyl acrylate, acrylamide, dimethylacrylamide, acrylonitrile, dimethylaminoethyl acrylate, dimethylaminoethyl methacrylate, tert-butylaminoethyl acrylate, tert-butylaminoethyl methacrylate, methoxyethyl acrylate, methoxyethyl methacrylate, glycidal methacrylate, and the like. In one embodiment, the polyacrylate is made from monomeric esters, such as monomeric esters of alcohols that have 1 to 8 carbon atoms in the alcohol. In certain embodiments, alcohols include alkyl alcohol, hydroxyalkyl alcohol, methoxyalkyl alcohol and vinyl alcohol. In certain embodiments, monomeric ester has only one such 1 to 8 carbon atoms group from an alcohol and one organic group from an organic acid (e.g., acrylic acid and methacrylic acid). Additional examples of appropriate acrylic adhesives suitable in the practice of the invention are described in Satas, “Acrylic Adhesives,” Handbook of pressure-Sensitive Adhesive Technology, 2nd ed., pp. 396-456 (D. Satas, ed.), Van Nostrand Reinhold, New York (1989). The acrylic adhesives are commercially available (National Starch and Chemical Corporation, Bridgewater, N.J.; Solutia, M A). Further examples of polyacrylate-based adhesives are as follows, identified as product numbers, manufactured by National Starch (Product Bulletin, 2000, DURO-TAK ® is a trademark of National Starch adhesives): 87-4098, 87-2287, 87-4287, 87-5216, 87-2051, 87-2052, 87-2054, 87-2196, 87-9259, 87-9261, 87-2979, 87-2510, 87-2353, 87-2100, 87-2852, 87-2074, 87-2258, 87-9085, 87-9301 and 87-5298. DURO-TAK ® 87-2287 and 87-4287 both are polymeric adhesives derived from monomer compositions that are similar: 5.2 wt % 2-hydroxyethyl acrylate, about 20-40 wt % vinyl acetate, and about 55-75 wt % 2-ethylhexyl acrylate; and these two polymeric adhesives are provided solubilized in ethyl acetate in solids content of about 40-50 wt %. The DURO-TAK ® 87-4287 monomeric components consist of the above-mentioned three monomeric esters. The DURO-TAK® 87-2287 adhesive is derived from monomeric components consisted of four monomers: vinyl acetate, 28%; 2-ethylhexyl acrylate, 67%; hydroxyethyl acrylate, 4.9%; and glycidyl methacrylate, 0.1%, see U.S. Pat. No. 5,693,335.

In certain embodiments, the adhesive in the reservoir layer has little or no acid functionality. In certain embodiments, it is substantially free of an adhesive polymer of acrylic acid or (meth) acrylic acid. In such adhesives, there is little or no adhesive that is polymerized from monomeric components of acrylic acid or (meth) acrylic acid. For example, the adhesive can have 4 wt % or less of a polymer that is polymerized from acrylic acid or (meth) acrylic acid monomers.

The polyacrylate material forming the reservoir layer has a solubility for the drug of about 0.5 wt % to about 15 wt % of the total polymer composition; preferably about lwt % to about l0 wt %; more preferably about 2 wt % to about 8 wt % of the total polymer composition 4. The reservoir layer, with or without the body-contacting adhesive, has a thickness of about 0.0125 mm (0.5 mil) to about 0.1 mm (4 mil); preferably about 0.018 mm (0.75 mil) to about 0.088 mm (3.5 mil); more preferably 0.023 mm (0.9 mil) to about 0.075 (3 mil); and even more preferably about 0.025 mm (1.05 mil) to about 0.05 mm (2 mil).

Such acrylates can also be used as an in-line body surface contacting (i.e., body-contacting) adhesive for attaching the device to the body surface during the use of the device. The thickness can be adjusted by one skilled in the art based on whether the acrylate body-contacting adhesive is to have a rate-controlling function.

In certain embodiments, the in-line body-contacting adhesive that can be used is a silicone adhesive. The silicone adhesives can be used as a reservoir layer for apixaban also, e.g., 4202 polydimethylsiloxane adhesive from Dow Corning. It is noted that other polysiloxane pressure sensitive adhesives can be used. Similar to the above in-line adhesives, the thickness can be adjusted by one skilled in the art based on whether the body-contacting adhesive is to have a rate-controlling function, in view of the present disclosure.

If desired, one can implement rate-control with a traditional “plastic” type of rate-control material such as polyolefin, e.g., polyethylene (high density, medium density or low density polyethylene, i.e., LDPE, etc.). However, in certain embodiments, for a desirable profile and ease of manufacturing, it is preferred that such plastic rate-control membrane is not used and/or the tie layer be implemented only to attach the layers together without imparting substantial rate-controlling function.

In one embodiment, the transdermal system can include a rate-control membrane placed between the reservoir layer and the body-contacting adhesive layer. Examples of rate-control membrane include but are not limited to EVA, high density polyethylene, and low density polyethylene. Examples of the types of polymer films that may be used to make the rate-control membrane are disclosed in U.S. Pat. Nos. 3,797,494 and 4,031,894, both of which are incorporated herein by reference. The above-mentioned EVA tie-layer can function as such a rate-control membrane for controlling flux rate of apixaban delivery.

Although no permeation enhancer (which includes absorption promoter) is needed in the present invention in that adequate apixaban flux can be achieved without permeation enhancer, if desired, permeation enhancer(s) can be used for further increasing the skin permeability of the drug apixaban or drug combinations to achieve delivery at therapeutically effective rates. Permeation enhancer(s) can be applied to the skin by pretreatment or currently with the drug, for example, by incorporation in the reservoir layer. A permeation enhancer should have the ability to enhance the permeability of the skin for one, or more drugs or other biologically active agents. A useful permeation enhancer would enhance permeability of the desired drug or biologically active agent at a rate adequate for therapeutic level from a reasonably sized patch (e.g., about 20 to 80 cm²).

Useful permeation enhancers include anionic surfactants (e.g. sodium lauryl sulfate, N-Lauryl Sarcosine, sodium octyl sulfate; cationic surfactants (e.g. cetyl trimethyl ammonium bromide, dodecyl pyridinium chloride, octyl trimethyl ammonium bromide); zwitterionic surfactants (like hexadecyl trimethyl ammoniopropane sulfonate, oleyl betaine, cocamidopropyl betaine); nonionic surfactants (e.g. polyoxyethylene sorbitan monolaurate (TWEEN20), sorbitan monolaurate, polyethyleneglycol dodecyl ether, Triton X-IOO); fatty acids (e.g. Oleic Acid, linoleic acid, linolenic acid); fatty esters (e.g. isopropyl myristate, sodium oleate, methyl laurate); azone/azone-like compounds (N-decyl-2-pyrrolidone, dodecyl amine, PP, nicotine sulfate); and others (e.g., menthol, methyl pyrrolidone, cineole, limonene). One or more permeation enhancers, alone or in combination, and which may include dissolution assistants, can constitute about 0 to 40% by weight, preferably about 0 to 30% by weight, and more preferably less than about 15% by weight solids of the resulting reservoir layer that has adequate pressure sensitive adhesive properties. In certain embodiments, the amount of permeation enhancers of about 15 wt % or less, preferably about 9 wt % or less, preferably about 5 wt % or less, and preferably none is used in the apixaban-containing reservoir layer. Also, although alkaline salts (e.g., sodium salts, potassium salts, ammonium salts, etc.) of organic acids such as acetic acid, lactic acid, citric acid, etc., can be used to increase apixaban absorption if desired, they are not necessarily used and in some embodiments, such organic salts are not used. Preferably about 10 wt % or less, preferably about 4 wt % or less of such salts (e.g., sodium acetate) is used. Although not needed, if it is desired to increase permeation and if any permeation enhancer is used, PVP is preferred, and no other permeation enhancers, such as fatty acids, alcohols, esters (such as esters of fatty acids) needs to be added. The PVP is preferably added into the in-line adhesive (e.g., PIB) and not in the drug reservoir layer.

### 5.5 ACRYLATE-BASED POLYMERS

One possible class of polymers for use in the apixaban transdermal patch of the present invention is acrylate-based polymers. Acrylate-based polymers adhere well to a variety of different surfaces and capable of being formulated to provide adhesive property.

Acrylate polymers may comprise copolymers of various monomers which may be “soft” monomers or “hard” monomers or combinations thereof. Soft monomers are characterized by having lower glass transition temperature. Examples of soft monomers include, but not limited to, n-butyl acrylate, 2-ethylhexyl acrylate and isooctyl acrylate. Hard monomers are characterized by having higher glass transition temperature. Examples of hard monomers include, but not limited to methyl methacrylate, ethyl acrylate and methyl acrylate. Soft monomers with lower glass transition temperature generally have higher solubility and better stability compared to hard monomers.

Monomers from which the acrylate polymers may be produced may comprise acrylic acid, methacrylic acid, butyl acrylate, butyl methacrylate, hexyl acrylate, hexyl methacrylate, isooctyl acrylate, isooctyl methacrylate, glycidyl methacrylate, 2-hydroxyethyl acrylate, methyl acrylate, methylmethacrylate, 2-ethylhexyl acrylate and 2-ethylhexyl methacrylate. Additional examples of acrylic adhesive monomers are described in Satas, “Acrylic Adhesives,” Handbook of Pressure-Sensitive Adhesive Technology, 2nd ed., pp. 396-456 (D. Satas, ed.), Van Nostrand Reinhold, New York (1989).

Acrylate polymers may comprise bipolymer, terpolymer or tetrapolymer or copolymers of even greater numbers of monomers, including copolymers of alkyl acrylates, alkyl methacrylates, coploymerizable secondary monomers and/or monomers having functional groups.

In addition, the acrylic-based polymers may have hydroxyl functional group and/or carboxyl functional groups which can influence properties of the polymers such as solubility of apixaban, miscibility with other components of the transdermal patch as well as apixaban flux rate.

In certain embodiments, acrylic-based polymers having functional groups are copolymers or terpolymers which contain monomer units having functional groups. The monomers can be monofunctional or polyfunctional. These functional groups include carboxyl groups, hydroxy groups, amino groups, amido groups, epoxy groups, etc. In certain embodiments, the functional groups are carboxyl groups and hydroxy groups. In certain embodiments, the carboxyl functional monomers include acrylic acid, methacrylic acid, itaconic acid, maleic acid, and crotonic acid. In certain embodiments, the hydroxy functional monomers include 2-hydroxyethyl methacrylate, 2-hydroxyethyl acrylate, hydroxymethyl acrylate, hydroxymethyl methacrylate, hydroxyethyl acrylate, hydroxyethyl methacrylate, hydroxypropyl acrylate, hydroxypropyl methacrylate, hydroxybutyl acrylate, hydroxybutyl methacrylate, hydroxyamyl acrylate, hydroxyamyl methacrylate, hydroxyhexyl acrylate, hydroxyhexyl methacrylate.

In certain embodiments, these functional monomers are incorporated into the copolymer or terpolymer in an amount of 0.1 to 20% by weight, 0.1 to 4% by weight, 4 to 8% by weight, based on the dry weight of the total acrylic-based polymer.

In certain embodiments, the proportions of acrylic-based polymers also depend on the content of the functional monomer units in the functional acrylic. In certain embodiments, a composition will require less of a functional acrylic that contains 20% by weight of functional groups as opposed to one that contains 0.5% by weight of functional groups to achieve the same effect required for solubility and flux. In certain embodiments, the amount of functional acrylic is within the range of about 1 to 99 weight %, 1 to 5 weight %, 5 to 20 weight %, or 20 to 30 weight %, 30 to 65% weight %, 65 to 99% weight %, based on the total polymer content of the composition. In certain embodiments, the amount of non-functional acrylic or acrylic with a functional group which does not have as great of an affinity for the drug, is within the range of about 99 to 1 weight %, 95 to 75 weight %, 75 to 65 weight %, or 65 to 30 weight %, 30 to 20 weight %, based on the total polymer content of the composition.

The acrylic-based polymers may or may not contain cross-linkers that provide chemical bonds between polymer chains so as to mitigate cold flow within the transdermal patch of the present invention. In some embodiments, the cross-linkers comprise about 0.01% to about 6% by weight of the drug-containing layer, or the reservoir layer. Examples of cross-linkers that may be used with acrylic-based polymers containing hydroxyl functional group include but are not limited to polybutyl titanate (PBT), tetrabutyl titanate (TBT), titanium dialkoxide bis(acetylacetonate) and/or titanium metal chelate. Examples of cross-linkers that may be used with acrylic-based polymers containing carboxyl functional group include but are not limited to aluminum tris(acetyl acetonate) and/or aluminium metal chelate. In addition, the acrylic-based polymers may be combined with tackifiers to provide adhesive property.

In certain embodiments, the carboxyl functional group containing acrylic-based polymer comprises an acrylate copolymer of 2-ethylhexyl acrylate, vinyl acetate, butyl acrylate, acrylic acid and a crosslinker.

In certain embodiments, the hydroxyl functional group containing acrylic-based polymer comprising an acrylate copolymer of 2-ethylhexyl acrylate, methyl acrylate and 2-hydroyxyethyl acrylate, or an acrylate copolymer of 2-ethylhexyl acrylate, vinyl acetate and 2-hydroxyethyl acrylate.

Examples of commercially available acrylic-based polymer that are acrylic-hydrocarbon hybrid polymers may be sourced from polymer solutions including, but not limited to, Duro-Tak™ 87-502B and Duro-Tak™ 87-504B, Duro-Tak™ 87-502A, Duro-Tak™ 87-503A and Duro-Tak™ 87-504A. Examples of acrylate-based polymers with no functional group may be sourced from polymer solutions including, but not limited to, Duro-Tak™ 87-4098, Duro-Tak™ 87-900A and Duro-Tak™ 87-9301. Examples of acrylate-based polymers having carboxyl functional group may be sourced from solutions including, but not limited to, Duro-Tak™ 87-235A (DT 235A), Duro-Tak™ 87-2353 (DT 2353), Duro-Tak™ 87-2852, Duro-Tak™ 87-2051, Duro-Tak™ 87-2052, Duro-Tak™ 87-2054 (DT 2054), Duro-Tak™ 87-2194 and Duro-Tak™ 87-2196. Examples of acrylate-based polymers having hydroxyl functional group may be sourced from solutions including, but not limited to Duro-Tak™ 87-2510 (DT 2510), Duro-Tak™ 87-2287, Duro-Tak™ 87-4287 and Duro-Tak™ 87-2516 (DT 2516). Examples of acrylate-based polymers having both hydroxyl and carboxyl functional groups may be sourced from solution including, but not limited to Duro-Tak™ 87-2074 and Duro-Tak™ 87-2979. In certain embodiment, the polymers are not Duro-Tak™ 387-2287, Duro-Tak™ 87-2287, Duro-Tak™ 87-900A, Duro-Tak™ 87-2194, Duro-Tak™ 287-2194 or Duro-Tak™ 87-2196.

In certain embodiments, the drug-containing layer, or the reservoir layer of the present dermal patch comprises one or more acrylates copolymers. In certain embodiments, the drug-containing layer, or the reservoir layer comprises a carboxyl functional group containing acrylic-based polymer which is an acrylates copolymer. In certain embodiments, the drug-containing layer, or the reservoir layer comprises a hydroxyl functional group containing acrylic-based polymer which is an acrylates copolymer.

Exemplary acrylic-based polymers and their properties are listed in Table 1.

TABLE 1

| | Typical Physical Properties | | | | |
|---|---|---|---|---|---|
| | | Contains | | | Viscosity |
| | Descrip- | vinyl | Contains | Solids | (cP or |
| Product | tion | acetate | Crosslinker | (%) | mPa-s) |
| Duro-Tak 87-900A | acrylates | No | n/a | 43 | 1800 |
| | copolymer | | | | |
| Duro-Tak 87-9301 | acrylates | No | n/a | 36.5 | 9500 |
| | copolymer | | | | |
| Duro-Tak 87-4098 | acrylates | Yes | n/a | 38.5 | 6500 |
| | copolymer | | | | |
| Duro-Tak 87-2510 | acrylates | No | No | 40.5 | 4250 |
| | copolymer | | | | |
| Duro-Tak 87-2287 | acrylates | Yes | No | 50.5 | 18000 |
| | copolymer | | | | |
| Duro-Tak 87-4287 | acrylates | Yes | No | 39 | 8000 |
| | copolymer | | | | |
| Duro-Tak 87-2516 | acrylates | Yes | Yes | 41.5 | 4350 |
| | copolymer | | | | |
| Duro-Tak 87-2074 | acrylates | No | Yes | 29.5 | 1500 |
| | copolymer | | | | |
| Duro-Tak 87-235A | acrylates | No | No | 36.5 | 8000 |
| | copolymer | | | | |
| Duro-Tak 87-2353 | acrylates | No | No | 36.5 | 8000 |
| | copolymer | | | | |
| Duro-Tak 87-2852 | acrylates | No | Yes | 33.5 | 2500 |
| | copolymer | | | | |
| Duro-Tak 87-2051 | acrylates | Yes | No | 51.5 | 4000 |
| | copolymer | | | | |
| Duro-Tak 87-2052 | acrylates | Yes | Yes | 47.5 | 2750 |
| | copolymer | | | | |
| Duro-Tak 87-2054 | acrylates | Yes | Yes | 47.5 | 2750 |
| | copolymer | | | | |
| Duro-Tak 87-2194 | acrylates | Yes | Yes | 45 | 3000 |
| | copolymer | | | | |
| Duro-Tak 87-2196 | acrylates | Yes | Yes | 45 | 2100 |
| | copolymer | | | | |
| Duro-Tak 87-2979 | acrylates | Yes | — | 44.5 | 2700 |
| | copolymer | | | | |
| Duro-Tak 87-2825 | acrylates | Yes | — | 47.5 | 1650 |
| | copolymer | | | | |
| Duro-Tak 87-2525 | acrylates | Yes | — | 41.5 | 4350 |
| | copolymer | | | | |

In certain embodiments, polymers with different characteristics may be combined to realize superior properties. In certain embodiments, the drug-containing layer, or the reservoir layer of the present transdermal patch (or the present composition) may comprise a combination of two or more polymers. In one embodiment, the two polymers may comprise two acrylate-based polymers. In certain embodiments, each polymer may comprise a carboxyl functional group, a hydroxyl functional group, or both functional groups.

### 5.5 ADDITIONAL FEATURES IN THE TRANSDERMAL DELIVERY SYSTEM

Transdermal delivery patches typically have protective layers or release liners. In one embodiment, the transdermal delivery system comprises a peelable protective layer (or liner). In one embodiment, the protective layer is made of a polymeric material that is metallized. Examples of the polymeric materials include polyurethane, polyvinyl acetate, polyvinylidene chloride, polypropylene, polycarbonate, polystyrene, polyethylene, polyethylene terephthalate, polybutylene terephthalate, paper, and the like, and a combination thereof. In certain embodiments, the protective layer includes a siliconized polyester sheet.

The backing layer can be made with conventional materials. It may be formed from any material suitable for making transdermal delivery patches, such as a breathable or occlusive material including fabric or sheet, made of polyvinyl acetate, polyvinylidene chloride, polyethylene, polyurethane, polyester, EVA, polyethylene terephthalate (PET), polybutylene terephthalate, coated paper products, aluminum sheet and the like, or a combination thereof. In preferred embodiments, the backing layer includes low density polyethylene (LDPE) materials, medium density polyethylene (MDPE) materials or high density polyethylene (HDPE) materials, e.g., SARANEX (Dow Chemical, Midland, Mich.). The backing layer may be a monolithic or a multilaminate layer. In preferred embodiments, the backing layer is a multilaminate layer including nonlinear LDPE layer/linear LDPE layer/nonlinear LDPE layer. A preferred backing material is a laminate of a thin occlusive PET or equivalent film tied to an EVA film. For a PET/EVA laminate backing, preferably the EVA side is facing the reservoir layer. Preferably the EVA in the backing contains 20 wt % or less of vinyl acetate content, more preferably about 12 wt % of vinyl acetate. Preferably the vinyl acetate content is about similar or within 5 wt % of the vinyl content (in wt %) of the tie layer (if any) that is disposed between the reservoir layer and any in-line adhesive. The backing layer can have a thickness of about 0.012 mm (0.5 mil) to 0.125 mm (5 mil); preferably about 0.025 mm (lmil) to 0.1 mm (4 mil); more preferably about 0.0625 mm (1.5 mil) to 0.0875 mm (3.5 mil).

To further improve body surface adhesion, optionally, an overlay adhesive can also be used. Typically an overlay is a layer of material positioned at the top (i.e., the side most distal from the body surface during application) of the device with adhesive on the body-proximal side of the overlay. The overlay has a size slightly larger in area than the reservoir layer in the device (which as a patch has a generally flat configuration) such that there is a ring-shaped overhang (or border) of the overlay around the device for the adhesive on the overhang to adhere securely to the body surface. An overlay can have an aggressive body-contacting adhesive, such as one with 16 wt % L100 PIB, 24 wt % OPPANOL B 12 PIB, 40 wt % INDOPOL H 1900 polybutene, and 20 wt % CRO SPOVIDONE. This adhesive is applied to a backing material, such as the 3M SCHOTCHPAK 9732 backing film previously described, or a non-woven elastomeric backing material. The overlay adhesive is cut to be larger than the active component of the patch (as described above), for example 2 cm longer in each linear dimension for a rectangular patch, and 2 cm longer in diameter for a circular patch. The overlay is laminated into place over the active component of the patch during manufacturing, held in place by the adhesive, and centered so as to provide, in this example, a lcm border around the patch perimeter for improving adhesion security of the system on the body surface.

Compatibility of the overlay adhesive and reservoir layer matrix material containing the drug can be further improved by interleaving a thin occlusive film there between. The transdermal drug delivery device comprises a drug reservoir layer disposed on a backing layer, a body-contacting adhesive, and a peelable (removable) protective layer (or release liner). The reservoir layer contains apixaban and optionally other drugs and the carrier material in the reservoir layer is suitable for carrying the pharmaceutical agents (or drugs) for transdermal delivery. Another adhesive layer is disposed on a backing forming an overlay more distal from the protective layer release liner.

To prevent the migration of apixaban from the reservoir layer to the adhesive of the overlay, a barrier frame with a shape resembling a window-frame for a rectangular patch or a “O” for a circular patch with internal dimensions smaller than that of the active area and outer dimensions greater than that of the area of the reservoir layer is placed between the reservoir layer and the overlay adhesive. In one embodiment, the barrier frame is disposed between the overlay adhesive layer and the backing layer of the drug reservoir layer. The reservoir layer has an outer perimeter (or edge) that is between the inner perimeter and outer perimeter of the barrier frame. The width (i.e., the distance between the inner perimeter and outer perimeter) of the barrier frame is selected, considering the cold flow characteristics and expected shelf life of the device, such that the barrier frame will prevent cold flow reservoir layer material to migrate past the outer perimeter to contact the overlay adhesive. As used herein, the term “between” means only that something is in a position intermediate two other things and does not necessarily mean that it is immediately adjacent to them or contacting them, unless specified to be the case. The border of the barrier frame that extends outside of the area of the reservoir layer is typically 1 to 5 mm, about 2-3 mm. Aligned between the active area and overlay adhesive, the interleaving frame ensures that even with some flow of adhesive or reservoir layer material from the active area during storage and wear, the adhesive from the overlay shall not come into contact with the reservoir layer material or adhesive proximal to the body surface and cause undesired drug migration.

The barrier material for making the backing layer can also be used for making the barrier frame. The barrier layer is impermeable to the drug in the reservoir layer; and preferably includes a material that is insoluble in water, alcohol and organic solvents. The barrier layer can be made from a polymer such as polyolefin laminates (Dow Chemical, Midland, Mich.), acrylonitrile copolymer films (BAREX, BP Chemicals, Koln, Germany), polyethylnapthalene (PEN), polyethylene terephthalate (PET), polyimide, polyurethane, polyethylene, polyvinyl acetate, polyvinylidene chloride, polybutylene terephthalate, coated paper products, metallized films and glass coated films where these films can include ethylene copolymers such as EVA, and combinations thereof. In preferred embodiments, the barrier layer contains polyester such as PET laminated to a polymer such as polyurethane, polyethylene, and ethylene copolymers. In preferred embodiments, the barrier layer contains polyester such as PET laminated to ethylene copolymers such as EVA. Other materials can be used, as long as the active agent or permeation enhancers are insoluble in them. The barrier layer as a single layer or as a multilaminate layer has a thickness of about 0.075 mm (0.3 mil) to about 0.125 mm (5mil); preferably about 0.025 mm (lmil) to about 0.1 mm (4 mil); more preferably about 0.0625 mm (1.5 mil) to about 0.0875 mm (3.5 mil); and even more preferably about 0.025 mm (lmil) to about 0.05 mm (2 mil).

Transdermal flux can be measured with a standard procedure using Franz cells or using an array of formulations. Flux experiments were done on isolated human cadaver epidermis. With Franz cells, in each Franz diffusion cell a disc of epidermis is placed on the receptor compartment. A transdermal delivery system is placed over the diffusion area (1.98 cm²) in the center of the receptor. The donor compartment is then added and clamped to the assembly. At time 0, receptor solution (between 21 cm and 24 ml, exactly measured) is added into the receptor compartment and the cell maintained at 35° C. This temperature yields a skin surface temperature of 30-32° C. Samples of the receptor compartment are taken periodically to determine the flux through skin and analyzed by HPLC. An alternative way to test flux is to use an array of patches. In testing flux with an array of transdermal miniature patches, formulations are prepared by mixing stock solutions of each of the mixture components of formulation in organic solvents (about 15 wt % solids), followed by a mixing process. The mixtures are then aliquoted onto arrays as 4-mm diameter drops and allowed to dry, leaving behind solid samples or “dots.” (i.e., mini-patches). The miniature patches in the arrays are then tested individually for flux through skin using a permeation array, whose principle of drug flux from a patch formulation through epidermis to a compartment of receptor medium is similar to that of Franz cells (an array of miniature cells). The test array has a plurality of cells, a piece of isolated human epidermis large enough to cover the whole array, and a multiple well plate with wells acting as the receptor compartments filled with receptor medium. The assembled permeation arrays are stored at 32° C. and 60% relative humidity for the duration of the permeation experiments. Receptor fluid is auto-sampled from each of the permeation wells at regular intervals and then measured by HPLC to determine the flux of the drug.

A wide variety of materials that can be used for fabricating the various layers of the transdermal delivery patches according to this invention have been described above. It is contemplated that materials other than those specifically disclosed herein, including those that may hereafter become known to the art to be capable of performing the necessary functions can be used by those skilled in the art.

### 5.6 ADMINISTRATION OF THE DRUG

On application to the skin, the drug in the drug reservoir layer of the transdermal patch diffuses into the skin where it is absorbed into the bloodstream to produce a systemic therapeutic effect. The onset of the therapeutic depends on various factors, such as, potency of the drug, the solubility and diffusivity of the drug in the skin, thickness of the skin, concentration of the drug within the body surface application site, concentration of the drug in the drug reservoir layer, and the like. On repeated sequential applications (by replacing a used patch with a new one), the residual drug in the application site of the patch is absorbed by the body at approximately the same rate that drug from the new patch is absorbed into the new application area.

Administration of the drug from a patch can be maintained for one day or a few days, e.g., at least three days, and up to seven days. It has been known in the past that beta blockers may tend to cause skin irritation or skin sensitization. See, e.g., B. F. O'DONNELL, I. S. FOULDS (1993) Contact allergy to beta-blocking agents in ophthalmic preparations Contact Dermatitis 28 (2), 121-122; Circulation. 1995 November; 1; 92(9):2526-39; and “Sensitization of human atrial 5-HT4 receptors by chronic beta-blocker treatment”, Sanders L, Lynham J A, Bond B, del Monte F, Harding S E, Kaumann A J; 1: Biol Pharm Bull. 1997 April; 20(4): 421-7.

Provided herein is a transdermal delivery device having a formulation that can deliver apixaban with no detectable irritation on skin. Additionally, the apixaban free base does not cause skin sensitization. As such, it can be considered to be “skin-compatible” or “biocompatible”.

### 5.7 PREPARATION OF TRANSDERMAL PATCH

The present transdermal patch may be formulated in accordance with procedures disclosed in, e.g., CN 103432,104; US publication 2016/0113908 and Tingting et al. AAPS Pharma SciTech (2016) published on line May 31, 2016, the disclosures of which are incorporated herein by reference.

In certain embodiments, the present transdermal patch may be made by preparing a blend of an appropriate amount of one or more polymer solutions such as Duro-TakTm 87-235A, Duro-Tak™ 87-2054, Duro-Tak™ 87-2510, and/or Duro-Tak™ 87-2516. These polymer solutions may comprise solvents such as DMI, DCM, ethyl acetate, heptane, n-heptane, hexane methanol, ethanol, isopropanol, 2,4-pentanedione, toluene, xylene or a combination thereof. Next, apixaban or a pharmaceutically acceptable salt thereof, permeation enhancers are added to the blend. In one embodiment, the blend has a viscosity of between about 0.1 to 18 Pascal seconds (Pa-s). The blend is then cast onto a release liner for drying at appropriate drying conditions to form the drug-containing layer, or the reservoir layer. During the drying process, the solvent(s) are evaporated so that only a trace remains. After the drying process, the drug-containing layer, or the reservoir layer is then laminated on one side onto a backing film while a release liner is applied onto the other side of the drug-containing layer, or the reservoir layer.

In certain embodiments, the transdermal devices are manufactured according to known methodology of laying adhesive on a backing and laminating different layers together. In one embodiment, a solution of the polymeric reservoir layer material is added to a double planetary mixer, followed by addition of desired amounts of the drug, and other ingredients that may be needed. Preferably, the polymeric reservoir layer material is an EVA hot melt material. Methods of hot melt processing and laminating to form transdermal devices are known to those skilled in the art. Generally, hot melt adhesives are processed at temperatures above room temperature, e.g., about or above 40° C., in melted condition to incorporate the drug and other excipients, and subsequently solidify to form the drug containing layer with adhesive and cohesive forces. The cohesive forces generally decrease with decreasing softening temperatures of the hot melt adhesives. Using plasticizers can lower the softening temperature of the hot melt material. A hot melt adhesive material, after melting and homogenizing with the desired drug at a temperature, can be spread onto a carrier material before cooling. A knife coating technique can be used to spread the hot melt mix on to a carrier surface for subsequently laminating with other layers.

### 5.8 USES OF TRANSDERMAL PATCH

On application to the skin, the apixaban in the matrix of the patch diffuses into the skin where it is absorbed into the bloodstream to produce a systemic drug effect. The onset of the drug effect depends on various factors, such as, potency of the apixaban, the solubility and diffusivity of the apixaban in the skin, thickness of the skin, concentration of the apixaban within the skin application site, concentration of the apixaban in the matrix, and the like. In one embodiment, the present transdermal patch is kept on the skin for about 12 hours to about 24 hours, about 20 hours to about 24 hours, or about 24 hours to about 30 hours, without removal. Then a new transdermal patch of the present invention is applied soon after to minimize fluctuations in apixaban blood concentration. In one embodiment, the apixaban patch is applied daily, twice weekly, or weekly. This is a good alternative for patients who have swallowing problems and using the patch, there is no need to crush the apixaban tablet. This will improve patient compliance and to reduce the risk of stroke and systemic embolism. Administration of apixaban via the patch also produce less plasma concentration variations and decrease the potential for GI bleeding and other side effects as compared to oral administration. The use of apixaban patch is also easy to terminate whereas oral administration does not allow easy termination of the drug.

### 5.9 CONDITIONS TO BE TREATED

The present disclosure provides methods and compositions (e.g., a transdermal patch, a topical composition, etc.) for treating or preventing thrombosis. In certain embodiments, the disclosure provides methods and compositions for treating or preventing left ventricular thrombus, atrial fibrillation, acute coronary syndrome, reduce the risk of stroke and systemic embolism. In one embodiment, the methods and compositions provide treatment of subjects with nonvalvular atrial fibrillation. In certain embodiments, the methods and compositions provide prophylaxis of deep vein thrombosis, which may lead to pulmonary embolism. In certain embodiments, the subject has undergone surgery. In certain embodiments, the methods and composition reduce the risk of recurrent deep vein thrombosis and pulmonary embolism following initial therapy.

### 5.10 COMBINATION WITH OTHER ACTIVE AGENTS

The present dermal patch containing the active agent (e.g., apixaban, or a pharmaceutically acceptable salt, derivative, or solvate thereof) or composition may be administered (or applied) to the subject simultaneously with, before, after, or in a sequence and within a time interval of, the administration of a second active agent(s).

By co-administration it is meant either the administration of a single composition containing both the present agent (e.g., apixaban, or a pharmaceutically acceptable salt, derivative, or solvate thereof) and a second active agent(s), or the administration of the present agent and a second active agent(s) as separate compositions within short time periods.

The present dermal patch or composition can be combined and administered with a second active agent(s) in separate compositions. In certain embodiments, the separate compositions are administered simultaneously. In certain embodiments, the separate compositions are not administered simultaneously, such as, for example, in a sequential manner.

The present dermal patch or composition may be administered (or applied) to a subject alone, or may be administered (or applied) to a subject in combination with one or more other treatments/agents (a second agent).

In certain embodiments, the second agent is an agent in the treatment of thrombosis or related disorders.

In certain embodiments, combination therapy means simultaneous administration of the compounds in the same composition, simultaneous administration of the compounds in separate compositions, or separate administration of the compounds (in separate compositions).

In certain embodiments, the second agent/treatment is used as adjunctive therapy to the present dermal patch or composition. In certain embodiments, the treatment includes a phase wherein treatment with the second agent/treatment takes place after treatment with the present dermal patch or composition has ceased. In certain embodiments, the treatment includes a phase where treatment with the present dermal patch or composition and treatment with the second agent/treatment overlap.

Combination therapy can be sequential or can be administered simultaneously. In either case, these drugs and/or therapies are said to be “co-administered.” It is to be understood that “co-administered” does not necessarily mean that the drugs and/or therapies are administered in a combined form (i.e., they may be administered separately (e.g., as separate compositions or formulations) or together (e.g., in the same formulation or composition) to the same or different sites at the same or different times).

In certain embodiments, a subject is treated concurrently (or concomitantly) with the present dermal patch or composition and a second agent. In certain embodiments, a subject is treated initially with the present dermal patch or composition, followed by cessation of the present dermal patch or composition treatment and initiation of treatment with a second agent. In certain embodiments, the present dermal patch or composition is used as an initial treatment, e.g., by administration of one, two or three doses, and a second agent is administered to prolong the effect of the present dermal patch or composition, or alternatively, to boost the effect of the present dermal patch or composition. A person of ordinary skill in the art will recognize that other variations of the presented schemes are possible, e.g., initiating treatment of a subject with the present dermal patch or composition, followed by a period wherein the subject is treated with a second agent as adjunct therapy to the present compound or composition treatment, followed by cessation of the present compound or composition treatment.

The present compound and the other pharmaceutically active agent(s) may be administered together or separately and, when administered separately this may occur simultaneously or sequentially in any order. The amounts of the present compound and the other pharmaceutically active agent(s) and the relative timings of administration will be selected in order to achieve the desired combined therapeutic effect.

In various embodiments, the therapies (e.g., a dermal patch or composition provided herein and a second agent in a combination therapy) are administered about 0 minutes to about 5 minutes apart, about 5 minutes to about 30 minutes apart, about 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 168 hours part. In certain embodiments, the therapies are administered no more than 24 hours apart or no more than 48 hours apart. In certain embodiments, two or more therapies are administered within the same patient visit. In other embodiments, the composition provided herein and the second agent are administered concurrently. In other embodiments, the composition provided herein and the second agent are administered at about 2 to 4 days apart, at about 4 to 6 days apart, at about 1 week part, at about 1 to 2 weeks apart, or more than 2 weeks apart. In certain embodiments, administration of the same agent may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same agent may be repeated and the administration may be separated by at least at least 1 day, 2 days, 3 days, 5 days, 10 days, 15 days, 30 days, 45 days, 2 months, 75 days, 3 months, or 6 months. In certain embodiments, a composition provided herein and a second agent are administered to a subject in a sequence and within a time interval such that the composition provided herein can act together with the other agent to provide an increased benefit than if they were administered otherwise. For example, the second active agent can be administered at the same time or sequentially in any order at different points in time; however, if not administered at the same time, they should be administered sufficiently close in time so as to provide the desired therapeutic or prophylactic effect. In one embodiment, the composition provided herein and the second active agent exerts their effect at times which overlap. Each second active agent can be administered separately, in any appropriate form and by any suitable route. In other embodiments, the composition provided herein is administered before, concurrently or after administration of the second active agent. In other embodiments, courses of treatment are administered concurrently to a patient, i.e., individual doses of the second agent are administered separately yet within a time interval such that the compound provided herein can work together with the second active agent. For example, one component can be administered once per week in combination with the other components that can be administered once every two weeks or once every three weeks. In other words, the dosing regimens are carried out concurrently even if the therapeutics are not administered simultaneously or during the same day.

The second agent can act additively or synergistically with the present agent/compound. In one embodiment, the composition provided herein is administered concurrently with one or more second agents in the same pharmaceutical composition. In another embodiment, a composition provided herein is administered concurrently with one or more second agents in separate pharmaceutical compositions. In still another embodiment, a composition provided herein is administered prior to or subsequent to administration of a second agent. Also contemplated are administration of a composition provided herein and a second agent by the same or different routes of administration, e.g., oral and parenteral. In certain embodiments, when the composition provided herein is administered concurrently with a second agent that potentially produces adverse side effects including, but not limited to, toxicity, the second active agent can advantageously be administered at a dose that falls below the threshold that the adverse side effect is elicited.

### 5.11 DOSING

The present dermal patch or composition may be administered (or applied) once, twice, three times, four times, five times, six times or more per day, or as needed, during the course of treatment. In certain embodiments, the present dermal patch or composition may be administered (or applied) at least once a day, at least twice a day, at least three times per day, or more. In certain embodiments, the present dermal patch or composition may be administered (or applied) at least once a week, at least twice a week, at least three times a week, at least once per month, at least twice per month, or more frequently. Treatment can continue as long as needed. In one embodiment, the dermal patch or composition may be administered (or applied) to a subject once daily.

The present dermal patch or composition may be administered (or applied) daily, weekly, biweekly, several times daily, semi-weekly, every other day, bi-weekly, quarterly, several times per week, semi-weekly, monthly etc., to maintain an effective dosage level. The duration and frequency of treatment may depend upon the subject's response to treatment.

In certain embodiments, a subject may be administered 1 dose, 2 doses, 3 doses, 4 doses, 5 doses, 6 doses or more of the present composition. In certain embodiments, a single dose of the present agent/composition is administered in the present method. In certain embodiments, multiple doses of the present agent/composition (e.g., 2 doses, 3 doses, 4 doses, 5 doses, 6 doses, 7 doses, 8 doses, 9 doses, 10 doses or more) are administered in the present method. In one embodiment, each dose equates to a single patch.

In certain embodiments, the administration of the present agent/composition is continued over a period of up to 2 days, up to 3 days, up to 4 days, up to 5 days, up to 6 days, up to 1 week, up to 2 weeks, up to 3 weeks, up to 4 weeks, 2 weeks, 3 weeks, 4 weeks, 5 weeks, 6 weeks, 7 weeks, 8 weeks, 9 weeks, 10 weeks, or longer.

In certain embodiments, the present agent/composition is administered once, twice, at least twice, at least three times, at least four times, at least five time, at least six times, at least seven times, at least eight times, at least nine times, or more per treatment.

### 5.12 SUBJECTS

The subject may be a human. In certain embodiments, the subject is a non-human animal. The non-human animal may be a mammal selected from the group consisting of primates (non-human primates), pigs, rodents, or rabbits. In an embodiment, the subject is a pig, such as a miniswine. In another embodiment, the subject is a mouse.

### 5.13 KITS

The present disclosure also encompasses an article of manufacture, e.g., a kit. The article of manufacture may contain the present dermal patch or composition in a suitable container with labeling and instructions for use. In certain embodiments, the container can be a dropper or tube with a suitable small orifice size, such as an extended tip tube made of any pharmaceutically suitable material. The topical formulations can be filled and packaged into a plastic squeeze bottle or tube. Optionally, an applicator can be provided in or attached to the container, or separately from the container.

Instructions may be packaged with the composition, for example, a pamphlet or package label. The labeling instructions explain how to the present composition, in an amount and for a period of time sufficient to treat or prevent the disorder or condition discussed herein. In certain embodiments, the label includes the dosage and administration instructions, the dermal patch's or topical formulation's composition, the clinical pharmacology, drug resistance, pharmacokinetics, absorption, bioavailability, and/or contraindications.

### 5.14 TOPICAL ADMINISTRATION

In certain embodiments, the present composition is formulated for topical administration. The terms “topically administrable composition,” a “topical composition,” or a “topical formulation,” as used herein, refer to any formulation or composition which is pharmaceutically and/or cosmetically acceptable for topical delivery of the specified compounds according to embodiments of the invention. The composition may be administered to a defined area of the body such as a defined area of skin surface or mucous membrane.

The present composition may additional contain a physiologically acceptable medium, such as a vehicle and/or a carrier. By “physiologically acceptable medium” is intended a cosmetically and/or dermatologically acceptable medium, which is compatible with the skin.

In some embodiments, the present composition can additionally include one or more pharmaceutically acceptable excipients. One of ordinary skill in the art would be familiar with pharmaceutically acceptable excipients. For example, the pharmaceutically acceptable excipient may be a water-soluble sugar, such as mannitol, sorbitol, fructose, glucose, lactose, and sucrose.

The present composition can be formulated in any pharmaceutical form normally provided for topical application to the skin, in particular formulated as solutions or dispersions of lotion or serum type, emulsions of liquid or semi-liquid consistency of the milk type, obtained by dispersion of a fatty phase in an aqueous phase (O/W) or, conversely, (W/O), or suspensions or emulsions of soft consistency of the aqueous or anhydrous cream or gel type, or, alternatively, microgranules, nanoparticles, microemulsions, nanocapsules, or vesicle dispersions of ionic and/or nonionic type.

Exemplary forms of formulation that can be used for topical administration include, but are not limited to, sprays, mists, aerosols, solutions, lotions, gels, serum, creams, ointments, pastes, unguents, emulsions and suspensions. The composition may be in the form of aqueous, aqueous/alcoholic or oily solutions, dispersions of lotion or serum type, aqueous anhydrous or lipophilic gels, emulsions of liquid or semi-liquid consistency of the milk type, obtained by dispersion of a fatty phase in an aqueous phase or conversely an aqueous phase in a fatty phase, or suspensions or emulsions of semi-solid or solid consistency of the cream or gel type, soaps or detergents, or alternatively microemulsions, microcapsules, microparticles, or vesicle dispersions of ionic and/or non-ionic type. Among additional alternative means for topical application of the compositions are spray pumps, aerosol dispersions, impregnated cosmetic facial masks, and impregnated cosmetic facial cloths or sponges.

In certain embodiments, the topically composition are prepared by mixing a pharmaceutically acceptable carrier with the present agent according to known methods in the art, for example, methods provided by standard reference texts such as, Remington: The Science and Practice of Pharmacy 1577-1591, 1672-1673, 866-885 (Alfonso R. Gennaro ed. 19th ed. 1995); Ghosh, T. K.; et al. Transdermal and Topical Drug Delivery Systems (1997), both of which are hereby incorporated herein by reference.

The present composition may contain a gelling agent, a polyol, a protective agent, a cosmetic agent, an adsorbent, a preservative, an antioxidant, a surfactant, a skin-penetration agent, a local anesthetic, an analgesic etc.

Suitable gelling agents known in the art, including those used in the two-phase or single-phase gel systems, can be used in the present invention. Some examples of suitable gelling agents are disclosed in Remington: The Science and Practice of Pharmacy 1517-1518 (Alfonso R. Gennaro ed. 19th ed. 1995), which is hereby incorporated herein by reference. The gelling agents include, but are not limited to, one or more hydrophilic and hydroalcoholic gelling agents used in the cosmetic and pharmaceutical industries. Non-limiting examples of gelling agents include hydroxyethylcellulose, cellulose gum, MVE/MA decadiene crosspolymer, PVM/MA copolymer, glycerine polyacrylate, or a combination thereof. Exemplary hydrophilic gelling agents include carboxyvinyl polymers (carbomer), acrylic copolymers such as acrylate/alkylacrylate copolymers, polyacrylamides, polysaccharides such as hydroxypropylcellulose, natural gums and clays, and, exemplary lipophilic gelling agents include modified clays such as bentones, metal salts of fatty acids such as aluminum stearates, and hydrophobic silica. Exemplary hydrophilic active agents are proteins or protein hydrolysates, amino acids, polyols, urea, allantoin, sugars and sugar derivatives, vitamins and hydroxy acids.

Polyols in gel formulations can serve one or more functions such as solubilizing agents, moisturizers, emollients, skin humectant, skin-penetration agents, etc. Suitable polyols that can be used in embodiments of the present invention include, but are not limited to, glycerine, propylene glycol, dipropylene glycol, hexylene glycol, butylene glycol, and liquid polyethylene glycols, such as polyethylene glycol 200 to 600. Other et al., Gels and Jellies, pp. 1327-1344 of Encyclopedia of Pharmaceutical Technology, vol. 3 (ed. by Swarbrick, et al, pub. by Marcel Dekker, 2002); or Pena, “Gel Dosage Forms: Theory, Formulation, and Processing,” pp. 381-388 of Topical Drug Delivery Formulations, (ed. by Osborne et al., pub. by Marcel Dekker, Inc., 1990).

Suitable preservatives include, but are not limited to, quaternary ammonium compounds, such as benzalkonium chloride, benzethonium chloride, cetrimide, dequalinium chloride, and cetylpyridinium chloride; alcoholic agents, for example, chlorobutanol, phenylethyl alcohol, and benzyl alcohol; parabens such as methylparaben, ethylparaben, propylparaben, and butylparaben; antibacterial esters, for example, esters of parahydroxybenzoic acid; and other anti-microbial agents such as chlorhexidine, chlorocresol, benzoic acid, polymyxin, and phenoxyethanol. Preferably, the preservative is selected from the group consisting of sodium benzoate, phenoxyethanol, benzyl alcohol, methylparaben, imidazolidinyl urea and diazolidinyl urea.

Topical administration can continue for about 1 day, about 2 days, about 3 days, about 4 days, about 5 days, about 6 days, about 7 days, about 2 weeks, about 3 weeks, about 4 weeks, about 2 months, about 3 months, about 4 months, about 5 months, about 6 months, about 1 year or longer.

In some embodiments, the present composition may comprise one or more pharmaceutically acceptable antioxidants. Any pharmaceutically acceptable antioxidant known to those of ordinary skill in the art is contemplated for inclusion in the present pharmaceutical compositions. For example, the pharmaceutically acceptable antioxidant may be selected from the group consisting of ascorbic acid, sodium ascorbate, sodium bisulfate, sodium metabisulfate and monothio glycerol.

In some embodiments, the present composition may comprise one or more pharmaceutically acceptable buffering agents. Any pharmaceutically acceptable buffering agent known to those of ordinary skill in the art is contemplated for inclusion in the present pharmaceutical compositions. Examples of such buffering agents include of monobasic sodium phosphate, dibasic sodium phosphate, sodium benzoate, potassium benzoate, sodium citrate, sodium acetate, and sodium tartrate.

The pH of the topical formulations may be within a physiologically acceptable pH, e.g., within the range of about 4 to about 8, of about 6 to about 7.5, or about 4.5 to 6.5.

In some embodiments, the present composition may or may not comprise one or more pharmaceutically acceptable skin penetration enhancers. Examples of such skin penetration enhancers include but not limited to fatty alcohols such as decanol, lauryl alcohol, linolenyl alcohol, n-octanol and oleyl alcohol; fatty acid esters such as ethyl acetate, dodecyl N,N-dimethylamino acetate, glycerol monolaurate, glycerol monooleate, isopropyl myristate, methyl laurate and sorbitan monooleate; fatty acids such as lauric acid and oleic acid; biologics such as lecithin, amines and amides such as N,N-dimethyl-m-toluamide, lauryl-amine and urea; complexing agents such as cyclodextrin, hydroxypropyl methylcellulose and liposomes; surfactants such as Brij 36T, sodium lauryl sulfate and sorbitan monooleate; other compounds such as dimethyl isosorbide, bisabolol, eucalyptol, menthol, terpenes, N-methyl pyrrolidone, azone, DMSO, MSM, decylmethyl sulfoxide, dimethyl formamide, dimethyl acetamide, glycols and propylene glycol.

Exemplary oils that may be used in the present composition, include mineral oils (liquid petroleum jelly), plant oils (liquid fraction of karite butter, sunflower oil), animal oils (perhydrosqualene), synthetic oils (purcellin oil), silicone oils (cyclomethicone) and fluoro oils (perfluoropolyethers). Fatty alcohols and fatty acids (stearic acid) can be added to these oils.

Exemplary emulsifiers that may be used in the present composition, include glyceryl stearate, polysorbate 60 and the mixture PEG-6/PEG-32/glycol stearate.

Representative solvents which can be used include the lower alcohols, such as ethanol and isopropanol.

In certain other embodiments, a surfactant can be used in the present composition, as a wetting agent, emulsifier, solubilizer and/or antimicrobial.

Suitable surfactants include, but are not limited to, sodium stearyl fumarate, diethanolamine cetyl sulfate, polyethylene glycol, isostearate, polyethoxylated castor oil, benzalkonium chloride, nonoxyl 10, octoxynol 9, polyoxyethylene sorbitan fatty acids (polysorbate 20, 40, 60 and 80), sodium lauryl sulfate, sorbitan esters (sorbitan monolaurate, sorbitan monooleate, sorbitan monopalmitate, sorbitan monostearate, sorbitan sesquioleate, sorbitan trioleate, sorbitan tristearate, sorbitan laurate, sorbitan oleate, sorbitan palmitate, sorbitan stearate, sorbitan dioleate, sorbitan sesqui-isostearate, sorbitan sesquistearate, sorbitan tri-isostearate), lecithin pharmaceutical acceptable salts thereof and combinations thereof.

In some embodiments, the topical formulations may contain moisturizing agents. Non-limiting examples of moisturizing agents that can be used with the compositions of the present invention include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturization factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol.

This invention will be better understood from the following examples. However, one skilled in the art will readily appreciate that the specific methods and results discussed are merely illustrative and not limiting.

In the example to follow, all parts and percentages are given by weight.

### 6. EXAMPLES

### Example 6.1

### Apixaban Solubility in Various Adhesive

1. Weight ˜4g of the adhesive individually,
2. Slower added ˜10-20 mg of apixaban into adhesive.
3. Mix the mixture overnight
4. Observe if apixaban had fully dissolved,
5. Repeat Step 2 to 4, if apixaban was fully dissolved
6. Stop when residual apixaban was observed

| | | Adhesive & 5% |
|---|---|---|
| Adhesive & 2% | Adhesive & 15% | erucic |
| Lactic acid | Acrylic acid | acid/DCM |
| 2510ₐ | 2510ₐ | 2516_{b} |
| 2516ₐ | 2516ₐ | 2287ₐ |
| 2287ₐ | 2287ₐ | 4287ₐ |
| 4287ₐ | 4287ₐ | 788ₐ |
| 788ₐ | 788ₐ | 2051ₐ |
| 2051ₐ | 2051_{b} | 2052ₐ |
| 2052ₐ | 2052ₐ | 2054ₐ |
| 2054ₐ | 2054_{c} | 235Aₐ |
| 235Aₐ | 235Aₐ | 2353ₐ |
| 2353_{c} | 2353ₐ | 2196ₐ |
| 2196ₐ | 2196ₐ | 2852ₐ |
| 2852ₐ | 2852_{b} | 2074ₐ |
| 2074ₐ | 2074ₐ | |
| 2979ₐ | 2979ₐ | |
| 900Aₐ | | |
| 9301ₐ | | |
| 4098ₐ | | |
| 9088ₐ | | |

ₐ= API Solubility < 1%
_{b}= API Solubility > 3%
_{c}= API Solubility > 5%

Apixaban solubility in various adhesives were tested. Apixaban does not dissolve in most of the adhesives. 235A, 2353 and 2852 provide some solubility. There is no turbidity. The wet adhesive mixture is clear but there are some particles.

### Example 6.2

Apixaban Solubility in Various Adhesives with Co-Solvent
1. Weight ˜5g of the adhesive individually,
2. ˜0.1 g of co-solvents were added into the adhesives and mix well
3. Added ˜10-20 mg of apixaban into adhesive-co-solvent mixture.
4. Mix the mixture overnight
5. Observe if apixaban had fully dissolved,
6. Repeat Step 3 to 5, if apxiaban was fully dissolved
7. Stop when residual apixaban was observed

| | | Adhesive & 5% |
|---|---|---|
| Adhesive & 2% | Adhesive & 15% | erucic |
| Lactic acid | Acrylic acid | acid/DCM |
| 2510ₐ | 2510ₐ | 2510 not tested |
| 2516ₐ | 2516ₐ | 2516_{b} |
| 2287ₐ | 2287ₐ | 2287ₐ |
| 4287ₐ | 4287ₐ | 4287ₐ |
| 788ₐ | 788ₐ | 788ₐ |
| 2051ₐ | 2051_{b} | 2051ₐ |
| 2052ₐ | 2052ₐ | 2052ₐ |
| 2054ₐ | 2054_{c} | 2054ₐ |
| 235Aₐ | 235Aₐ | 235Aₐ |
| 2353ₐ | 2353ₐ | 2353ₐ |
| 2196ₐ | 2196ₐ | 2196ₐ |
| 2852ₐ | 2852_{b} | 2852ₐ |
| 2074ₐ | 2074ₐ | 2074ₐ |
| 2979ₐ | 2979ₐ | |
| 900Aₐ | | |
| *9301_{c} | | |
| *4098_{c} | | |
| 9088ₐ | | |

ₐ= API Solubility < 1%
_{b}= API Solubility > 3%
_{c}= API Solubility > 5%

Different co-solvent with different adhesives provide different results but the solubility is still low. Selection of more than two co-solvents will increase the solubility. However, solubility does not equal better permeation.

### Example 6.3

### Preliminary Permeation Studies

Flux rate of the apixaban transdermal patch of the present invention is measured with a standard procedure using Franz diffusion cells and human cadaver skin as described in Strasinger C., Raney S., Tran D., Ghosh P., Newman B., Bashaw E., Ghosh T. and Shukla C., Navigating sticky areas in transdermal product development, Journal of Controlled Release: 233(2016) 1-9.

In each Franz diffusion cell a disc (diameter of 25 mm) of human cadaver skin is placed on the receptor compartment. A transdermal delivery system is cut the same size as the skin and placed over the diffusion area in the center of the receptor. The donor compartment is then added and clamped to the assembly. At time 0, receptor medium solution 14 mL is added into the receptor compartment and the cell maintained at 32+2° C. Samples of the receptor compartment are taken periodically to determine the skin flux and analyzed by HPLC. The apixaban concentration in the sampled solution was assayed by HPLC, and the flux value (value of the skin permeation rate of the drug in a steady state) and cumulative permeation were calculated.

| | Drug Composition (% w/w) | | | |
|---|---|---|---|---|
| Ingredients | Exam 1 | Exam 2 | Exam 3 | Exam 4 |
| Apixaban | 3 | 3 | 3 | 5 |
| Lactic acid | 10 | 10 | 10 | 10 |
| Stearic acid | 3 | 3 | 3 | |
| Methyl-1-lactate | 5 | 5 | 5 | |
| Acrylic aicd | | | | 3 |
| Durotak 87-235A | 79 | | | 82 |
| GMS 788 | | 79 | | |
| Durotak 87-2516 | | | 79 | |
| Hour | Average Cumulative Amount (μg/cm²) | | | |
| 2 | 0.00 | 0.17 | 0.00 | 0.00 |
| 4 | 0.00 | 0.19 | 0.00 | 0.78 |
| 6 | 0.00 | 0.22 | 0.05 | 1.39 |
| 8 | 0.00 | 0.24 | 0.05 | 1.88 |
| 24 | 0.40 | 3.10 | 9.81 | 8.64 |
| 26 | 0.60 | 4.15 | 13.27 | 10.26 |
| Flux (μg/cm² hr) | 0.024 | 0.167 | 0.560 | 0.419 |

OH-functional adhesive has better permeation and release of the API, still there is no penetration.

No differences between the two —OH functional adhesives (GMS 788 and DT 2516).

### Example 6.4

### Solution Permeation Studies

In each Franz diffusion cell a disc (diameter of 25 mm) of human cadaver skin is placed between the donor and the receptor compartment. At time 0, receptor medium solution 14 mL is added into the receptor compartment and the cell maintained at 32±2° C. The solution is added into the donor compartment. Samples of the receptor compartment are taken periodically to determine the skin flux and analyzed by HPLC. The apixaban concentration in the sampled solution was assayed by HPLC, and the flux value (value of the skin permeation rate of the drug in a steady state) and cumulative permeation were calculated.

Average cumulative amount (μg/cm²) of different formulation

| | Average Cumulative Amount | | | | | | Flux |
|---|---|---|---|---|---|---|---|
| Formulation | (μg/cm²) | | | | | | (μg/ |
| Information | 2 | 4 | 6 | 8 | 24 | 26 | cm² hr) |
| API Super Saturated | 0.60 | 1.46 | 2.25 | 2.94 | 10.90 | 21.05 | 0.93 |
| in DMSO | | | | | | | |
| API Super Saturated | 0.00 | 0.38 | 0.73 | 1.02 | 5.21 | 6.20 | 0.26 |
| in DCM Stearic Acid | | | | | | | |
| API Super Saturated | 2.92 | 8.18 | 13.33 | 18.15 | 43.55 | 44.90 | 1.71 |
| in Acrylic Acid | | | | | | | |

In vitro permeation study was conducted on two different human cadaver skin. Apixaban in DMSO is the positive control, Apixaban in DCM/stearic acid is as the negative control. Apixaban with Enhancer shows 2 times higher permeated amount to Apixaban in DMSO. In the presence of an enhancer, the apixaban permeate through the human skin.

### Example 6.5

### Preliminary Permeation Studies

In each Franz diffusion cell a disc (diameter of 25 mm) of human cadaver skin is placed between the donor and the receptor compartment. At time 0, receptor medium solution 14 mL is added into the receptor compartment and the cell maintained at 32±2° C. The solution is added into the donor compartment. Samples of the receptor compartment are taken periodically to determine the skin flux and analyzed by HPLC.

The apixaban concentration in the sampled solution was assayed by HPLC, and the flux value (value of the skin permeation rate of the drug in a steady state) and cumulative permeation were calculated.

Average cumulative amount (μg/cm²) of different formulation

| Formulation No. | 2 | 4 | 6 | 8 | 24 | 32 | 48 |
|---|---|---|---|---|---|---|---|
| 1,2- | 19.65 | 39.09 | 50.66 | 53.84 | 86.66 | 97.77 | 130.78 |
| propyleneglycol/ | | | | | | | |
| lactic acid | | | | | | | |
| Caprylocaproyl | 0.03 | 5.92 | 2.40 | 3.79 | 10.53 | 19.50 | 35.12 |
| Polyoxyl-8- | | | | | | | |
| glycerides/ | | | | | | | |
| lactic acid | | | | | | | |
| Polyethylene | 0.00 | 0.04 | 0.08 | 0.62 | 6.74 | 13.92 | 32.09 |
| glycol | | | | | | | |
| dodecyl ether/ | | | | | | | |
| lactic acid | | | | | | | |
| Tween 20/ | 0.00 | 0.07 | 0.30 | 0.57 | 3.07 | 4.12 | 6.95 |
| lactic acid | | | | | | | |
| Tween 80/ | 0.00 | 0.18 | 0.39 | 0.60 | 2.46 | 3.69 | 7.24 |
| lactic acid | | | | | | | |
| 1,2- | 0.16 | 1.07 | 2.15 | 3.04 | 5.70 | 7.09 | 12.64 |
| propyleneglycol | | | | | | | |

In vitro permeation study showed that combination of 1,2-propyleneglycol/lactic acid shows superior penetration than other combinations. 1,2-propyleneglycol alone did not show unexpected effect.

Various formulations were prepared according to the transdermal patch preparation procedures described herein.

### Example 6.5.1

### Permeation Studies on Topical Solution Formulation

| | Drug Composition in Solution | | | |
|---|---|---|---|---|
| | (% w/w) | | | |
| Ingredients | Exam 5 | Exam 6 | Exam 7 | Exam 8 |
| Apixaban | 0.5 | 3.9 | 0.8 | 0.8 |
| Lactic acid | 9.9 | 13.9 | | |
| Kollisolv PG | | 28.3 | 58.4 | 58.3 |
| Labrasol | 56.0 | | | |
| DCM | 33.6 | | | |
| Chloroform | | 53.9 | 40.8 | 40.7 |
| Flux | 0.7 | 2.2 | 0.1 | 0.4 |
| (μg/cm² hr) | | | | |

In vitro permeation study was conducted on 5 different human cadaver skin (leg) and the receptor fluid is Sodium phosphate with 0.5% SLS buffer pH 6.5 at 32+1° C.

In combination of 1,2-propyleneglycol and lactic acid shows superior penetration than other combinations.

### Example 6.5.2

### Permeation Studies on Hydrogel Formulation

| | Drug Composition (% w/w) | | | |
|---|---|---|---|---|
| Ingredients | Exam 9-1 | Exam 9-2 | Exam 10 | Exam 11 |
| Apixaban | 3.21 | 3.19 | 3.12 | 4.05 |
| PPG | 25.67 | 25.73 | 24.99 | 26.96 |
| Latic acid | 25.56 | 13.70 | 12.73 | |
| Eugenol | 6.27 | 25.53 | 24.89 | 28.43 |
| HPC_{-L} | 13.35 | 6.39 | 6.21 | 6.77 |
| Glycerin | | | 2.46 | 2.95 |
| MeOH | 25.94 | 25.46 | 25.61 | |
| DCM | | | | 30.85 |
| Flux | 2.41 | 7.11 | 3.50 | 0.97 |
| (μg/cm² hr) | | | | |

### Effect of HPCL and Glycerin:

with lower amount of HPC higher the flux;

without glycerin higher the flux,

without lactic acid, lower the flux

### Example 6.5.3

Permeation Studies on COOH Function group acrylic matrix patch formulation

| | Drug Composition | | |
|---|---|---|---|
| | (% w/w) | | |
| Ingredients | Exam 12 | Exam 13 | Exam 14 |
| Apixaban | 3.51 | 3.67 | 3.67 |
| Acrylic acid | 17.54 | 18.35 | 18.35 |
| Lactic acid | 17.54 | 18.35 | 18.35 |
| Octanoic acid | 17.54 | 13.76 | 13.76 |
| Tween 20 | 8.77 | | |
| Brij L 4 | | 9.17 | |
| Span 80 | | | 9.17 |
| Durotak 87-2196 | 35.09 | 36.70 | 36.70 |

| | Drug Composition | | |
|---|---|---|---|
| | (% w/w) | | |
| Ingedients | Exam 15 | Exam 16 | Exam 17 |
| Apixaban | 3.51 | 3.51 | 3.51 |
| Acrylic acid | 17.54 | 17.54 | 17.54 |
| Lactic acid | 17.54 | 17.54 | 17.54 |
| Octanoic acid | 17.54 | 17.54 | 17.54 |
| Eugenol | 8.77 | | |
| Triacetin | | 8.77 | |
| Methyl-1-lactate | | | 8.77 |
| Durotak 87-2353 | 35.09 | 35.09 | 35.09 |

### Example 6.5.4

Permeation studies on OH functional group acrylic matrix patch formulation

| | Drug Composition (% w/w) | | | | |
|---|---|---|---|---|---|
| Ingredients | Exam 18 | Exam 19 | Exam 20 | Exam 21 | Exam 22 |
| Apixaban | 3.67 | 3.67 | 3.42 | 3.42 | 3.42 |
| Acrylic acid | 16.51 | 16.51 | 18.80 | 18.80 | 18.80 |
| Lactic acid | 16.51 | 16.51 | 18.80 | 18.80 | 18.80 |
| Octanoic acid | 13.76 | 13.76 | 12.82 | 12.82 | 12.82 |
| Span 80 | 9.17 | 9.17 | 8.55 | 8.55 | |
| Eugenol | 3.67 | | | | 3.42 |
| Methyl-1-lactate | | 3.67 | | | 8.55 |
| Labrasol | | | | | |
| Eucalyptol | | | 3.42 | | |
| Lauryl lactate | | | | 3.47 | |
| Durotak 87-2510 | 36.70 | 36.70 | 34.19 | 34.19 | 34.19 |

### Examples 6.5.5

### Permeation Studies on Formulation for 7-Day Matrix Patch

| | Drug Composition (% w/w) | | | |
|---|---|---|---|---|
| Ingredients | Exam 12-1 | Exam 14-1 | Exam 12-2 | Exam 14-2 |
| Apixaban | 3.51 | 3.67 | 3.39 | 3.39 |
| Acrylic acid | 17.54 | 18.35 | 18.64 | 18.64 |
| Lactic acid | 17.54 | 18.35 | 18.64 | 18.64 |
| Octanoic acid | 17.54 | 13.76 | 16.95 | 16.95 |
| Tween 20 | 8.77 | | 8.47 | |
| Brij L 4 | | | | |
| Span 80 | | 9.17 | | 8.47 |
| Durotak 87-2196 | 35.09 | 36.70 | 33.90 | 33.90 |
| Flux (μg/cm² hr) | 1.71 | 1.88 | 3.22 | 3.36 |

### Exemplary Systems and Methods are Set Out in the Following Items:

Item 1. A transdermal patch comprising: (i) a drug-containing layer; (ii) a matrix layer or a reservoir layer and (iii) a backing layer, wherein the drug-containing layer comprises apixaban or a pharmaceutically acceptable salt thereof and wherein the transdermal patch has an average input rate of about 0.5-20 μg/cm2hr.
Item 2. The transdermal patch of Item 1 wherein the matrix layer or reservoir layer comprises an acrylic-based polymer, wherein the acrylic-based polymer comprises a hydroxyl functional group containing acrylic-based polymer.
Item 3. The transdermal patch of any of the preceding Items wherein the matrix layer or reservoir layer further comprises a silicone adhesive.
Item 4. The transdermal patch of any of the preceding items wherein the matrix layer or reservoir layer comprises a polyisobutylene polymer, silicone, EVA polymer, non-acidic polyacrylate, hydrogel polymer or a combination thereof.
Item 5. The transdermal patch of any of the preceding items, wherein the acrylic-based polymer and the silicone are at a weight ratio ranging from about 5:1 to about 1:5.
Item 6. The transdermal patch of any of the preceding items, wherein the hydroxyl functional group containing acrylic-based polymer is an acrylates copolymer.
Item 7. The transdermal patch of any of the preceding items, wherein the acrylic-based polymers provides a solubility of greater than 5% for apixaban or a pharmaceutically acceptable salt thereof.
Item 8. The transdermal patch of any of the preceding items wherein the matrix layer or reservoir layer comprises one or more solvents.
Item 9. The transdermal patch of any of the preceding items wherein the solvent is lactic acid, acrylic acid, erucic acid or a combination thereof.
Item 10. The transdermal patch of any of the preceding items wherein the solvent is 1,2-propyleneglycol, lactic acid or a combination thereof.
Item 11. The transdermal patch of any of the preceding items, wherein the apixaban or a pharmaceutically acceptable salt thereof is in an amount ranging from about 0.1% to about 15% by weight (wt %) relative to total weight of the drug-containing layer.
Item 12. The transdermal patch of any of the preceding items, wherein the hydroxyl functional group containing acrylic-based polymer is sourced from a polymer solution of acrylates copolymer comprising 2-hydroxyethyl acrylate or from a polymer solution of acrylates copolymer comprising vinyl acetate and 2-hydroxyethyl acrylate.
Item 13. The transdermal patch of any of the preceding items, wherein the drug-containing layer further comprises a permeation enhancer.
Item 14. The transdermal patch of item 13, wherein the permeation enhancer is an alcohol, a fatty acid, a fatty alcohol, a pharmaceutically acceptable solvent, a pharmaceutically acceptable surfactant, or combinations thereof.
Item 15. The transdermal patch of item 13, wherein the permeation enhancer is selected from the group consisting of aliphatic alcohols, fatty acids having chain of 8 to 20 carbons, fatty acid esters, alcohol amines, polyhydric alcohol alkyl ethers, polyoxyethylene alkyl ethers, glycerides, middle-chain fatty acid esters of polyhydric alcohols having chain of 8-20 carbon atoms, alkyl esters having chain of 1-6 carbon atoms, acylated amino acids, pyrrolidone, pyrrolidone derivatives, ethoxylated fatty alcohols, pharmaceutically acceptable surfactants or a combination thereof.
Item 16. The transdermal patch of items 13-15, wherein the permeation enhancer is 1,2-propyleneglycol, a polysorbate, hydroxypropyl cellulose (HPC), or combinations thereof.
Item 17. The transdermal patch of items 13-16, wherein the permeation enhancer comprises polysorbate 80.
Item 18. The transdermal patch of items 13-17, wherein the permeation enhancer is in an amount ranging from about 5% to about 30% by weight (wt %) relative to total weight of the drug-containing layer.
Item 19. The transdermal patch of items 13-18, wherein the permeation enhancer provides a solubility of greater than about 20 mg/mL for apixaban or a pharmaceutically acceptable salt thereof.
Item 20. The transdermal patch of any of the preceding items, further comprising an organic solvent.
Item 21. The transdermal patch of items 20, wherein the organic solvent is 1,3-Dimethyl-2-imidazolidinone (DMI), dichloromethane (DCM), or a combination thereof.
Item 22. The transdermal patch of items 9, wherein the solvent is in an amount ranging from about 5% to about 30% by weight (wt %) relative to total weight of the drug-containing layer.
Item 23. The transdermal patch of any of the preceding items, wherein the drug-containing layer further comprises a crystallization inhibitor.
Item 24. The transdermal patch of any of the preceding items, wherein the drug-containing layer further comprises an antioxidant.
Item 25. The transdermal patch of any of the preceding items, further comprising a protective layer.
Item 26. The transdermal patch of any of the preceding items, wherein the transdermal patch provides a flux rate of about 0.5 μg/cm2.hr to about 20 μg/cm2.hr for up to about 30 hours.
Item 27. The transdermal patch of any of the preceding items, wherein lag time for the transdermal patch is less than about 8 hours.
Item 28. The transdermal patch of any of the preceding items wherein the patch size is from 4 cm 2 to 40 cm 2 and the rate-control in-line adhesive is one of polyisobutylene (PIB), silicone, and polyacrylate that controls the delivery rate of apixaban at 4 to 12 mg per day.
Item 29. The transdermal patch of any of the preceding items wherein the apixaban is dissolved or dispersed in a hot melt adhesive in the reservoir layer.
Item 30. The transdermal patch of any of the preceding items wherein the reservoir layer includes about 5-15 wt % of permeation enhancer.
Item 31. The transdermal patch of any of the preceding items wherein the apixaban is dissolved in a hot melt of EVA in the reservoir layer, the device having an in-line rate-control adhesive of PIB.
Item 32. The transdermal patch of any of the preceding items comprising a rate-control in-line adhesive different from the main matrix polymer, and the patch further comprising an EVA tie layer disposed between the reservoir layer and the in-line adhesive.
Item 33. The transdermal patch of any of the preceding items wherein the reservoir layer contains EVA with 10-80 wt % or more of vinyl acetate content.
Item 34. The transdermal patch of any of the preceding items wherein the reservoir layer contains apixaban free base adequate for delivery for 1-3 days.
Item 35. The transdermal patch of any of the preceding items wherein the reservoir layer contains apixaban free base adequate for delivery for 3-9 days.
Item 36. The transdermal patch of any of the preceding items wherein the reservoir layer contains 20-30 wt % apixaban free base and 30-60 wt % EVA.
Item 37. The transdermal patch of any of the preceding items wherein the reservoir layer is substantially free of an adhesive polymer with acidic functionality.
Item 38. The transdermal patch of any of the preceding items wherein the device has an average apixaban flux of 5-25 mcg/(cm 2.h) for 1 day.
Item 39. The transdermal patch of any of the preceding items wherein the reservoir layer contains apixaban free base adequate for delivery for 3 days or more with an average flux of a flux of 5-25 mcg/(cm 2.h).
Item 40. The transdermal patch of any of the preceding items further comprising an adhesive overlay disposed distal to the backing layer and a protective liner disposed proximal to the in-line rate-control adhesive.
Item 41. The transdermal patch of any of the preceding items wherein the rate-control adhesive is silicone adhesive.
Item 42. The transdermal patch of any of the preceding items wherein the rate-control adhesive contains 1-4 wt % of an alkaline salt of an organic acid.
Item 43. A method for making a transdermal patch for administering a apixaban to a user, comprising: (a) disposing a reservoir layer proximally relative to a backing layer; and (b) forming the reservoir layer, which contains a polymeric composition containing an amount of apixaban free base sufficient for multiple-day delivery.
Item 44. The method of item 43 comprising including an in-line rate-control adhesive more proximal to the user's body surface relative to the reservoir layer.
Item 45. The method of any of items 43 to 44 comprising using a patch size from 4 cm2 to 40 cm2 including polyisobutylene (PIB) as the in-line rate-control adhesive such that the PIB controls the delivery rate of apixaban free base at 4 to 12 mg per day.
Item 46. The method of any of items 43 to 45 comprising dissolving or dispersing the apixaban free base in a hot melt of poly(ethylene-co-vinyl acetate) (EVA) for the reservoir layer, further comprising disposing a tie layer between the reservoir layer and in-line rate-control adhesive.
Item 47. The method of any of items 43 to 46 comprising dissolving or dispersing the apixaban free base in a hot melt of EVA for the reservoir layer, wherein the backing layer contains EVA, further comprising disposing an EVA tie layer between the reservoir layer and the in-line rate-control adhesive that comprises PIB.
Item 48. The method of any of items 43 to 47 wherein the backing layer has EVA and the tie layer EVA and the backing layer EVA both have lower vinyl acetate content than the EVA in the reservoir layer.
Item 49. The method of any of items 43 to 48 wherein the backing layer has EVA and the tie layer EVA and the backing layer EVA both have lower vinyl acetate content than the EVA in the reservoir layer, the reservoir layer EVA having 20-50 wt % vinyl acetate.
Item 50. The method of any of items 43 to 49 comprising including apixaban free base in the reservoir layer adequate for delivery for 1 to 7 days.
Item 51. The method of items 43 to 50 comprising including in the reservoir layer 20-50 wt % apixaban free base and 60-80 wt % EVA.
Item 52. The transdermal patch of items 43 to 51 wherein the reservoir layer is substantially free of an adhesive polymer with acid functionality.
Item 53. A method of treating thrombosis or related disorder in a subject comprising administering a transdermal delivery patch, wherein the transdermal delivery patch comprising: (a) backing layer; and (b) a matrix layer or a reservoir layer disposed proximally relative to the backing layer, said matrix layer or reservoir layer comprising a polymeric composition containing an amount of apixaban free base sufficient for multiple-day delivery.
Item 54. A method for treating thrombosis or a related disorder comprising the step of applying the transdermal patch of any of the preceding items to a human subject in need thereof.
Item 55. The method of any of the preceding items, wherein the transdermal patch is applied to the human subject for a period of about 24 hours.
Item 56. The method of any of the preceding items, wherein about 1 mg to about 80 mg of apixaban is delivered from the transdermal patch to the human subject daily.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Patents, patent applications, and publications are cited throughout this application, the disclosures of which, particularly, including all disclosed chemical structures, are incorporated herein by reference. Citation of the above publications or documents is not intended as an admission that any of the foregoing is pertinent prior art, nor does it constitute any admission as to the contents or date of these publications or documents. All references cited herein are incorporated by reference to the same extent as if each individual publication, patent application, or patent, was specifically and individually indicated to be incorporated by reference.

The foregoing written specification is considered to be sufficient to enable one skilled in the art to practice the invention. Various modifications of the invention in addition to those shown and described herein will become apparent to those skilled in the art from the foregoing description and fall within the scope of the appended claims.

1-56. (canceled)

57. A transdermal patch comprising: (i) a drug-containing layer; and (ii) a backing layer, wherein the drug-containing layer comprises apixaban or a pharmaceutically acceptable salt thereof and wherein the transdermal patch has an average input rate of about 0.5-20 μg/cm2hr.

58. The transdermal patch of claim 57 wherein the drug-containing layer is a matrix layer or a reservoir layer.

59. The transdermal patch of claim 57 further comprises a matrix layer or a reservoir layer.

60. The transdermal patch of claim 58 wherein the matrix layer or reservoir layer comprises an acrylic-based polymer, wherein the acrylic-based polymer comprises a hydroxyl functional group containing acrylic-based polymer.

61. The transdermal patch of claim 58 wherein the matrix layer or reservoir layer further comprises a silicone adhesive.

62. The transdermal patch of claim 58 wherein the matrix layer or reservoir layer comprises a polyisobutylene polymer, silicone, EVA polymer, non-acidic polyacrylate, hydrogel polymer or a combination thereof.

63. The transdermal patch of claim 60, wherein the hydroxyl functional group containing acrylic-based polymer is an acrylates copolymer.

64. The transdermal patch of claim 60, wherein the acrylic-based polymers provides a solubility of greater than 3% for apixaban or a pharmaceutically acceptable salt thereof.

65. The transdermal patch of claim 58 wherein the matrix layer or reservoir layer comprises one or more solvents.

66. The transdermal patch of claim 57, wherein the apixaban or a pharmaceutically acceptable salt thereof is in an amount ranging from about 0.1% to about 15% by weight (wt %) relative to total weight of the drug-containing layer.

67. The transdermal patch of claim 60, wherein the hydroxyl functional group containing acrylic-based polymer is sourced from a polymer solution of acrylates copolymer comprising 2-hydroxyethyl acrylate or from a polymer solution of acrylates copolymer comprising vinyl acetate and 2-hydroxyethyl acrylate.

68. The transdermal patch of claim 57, wherein the drug-containing layer further comprises a permeation enhancer.

69. The transdermal patch of claim 68, wherein the permeation enhancer is an alcohol, a fatty acid, a fatty alcohol, a pharmaceutically acceptable solvent, a pharmaceutically acceptable surfactant, or combinations thereof.

70. A method for making a transdermal patch for administering a apixaban to a user, comprising: (a) disposing a reservoir layer proximally relative to a backing layer; and (b) forming the reservoir layer, which contains a polymeric composition containing an amount of apixaban free base sufficient for multiple-day delivery.

71. The method of claim 70 comprising including an in-line rate-control adhesive more proximal to the user's body surface relative to the reservoir layer.

72. A method of treating thrombosis or related disorder in a subject comprising administering a transdermal delivery patch, wherein the transdermal delivery patch comprising:
(a) backing layer; and (b) a matrix layer or a reservoir layer disposed proximally relative to the backing layer, said matrix layer or reservoir layer comprising a polymeric composition containing an amount of apixaban free base sufficient for multiple-day delivery.

73. A method for treating thrombosis or a related disorder comprising the step of applying claim 57 to a human subject in need thereof.

74. The method of claim 73, wherein the transdermal patch is applied to the human subject for a period of more than 24 hours, 1 day to 3 days, 3 days to 4 days, or 4 days to 7 days.

19.

75. The method of claim 73, wherein about 1 mg to about 80 mg of apixaban is delivered from the transdermal patch to the human subject daily.

76. The transdermal patch of claim 73 wherein the drug-containing layer is a matrix layer or a reservoir layer.
